SEQUENCE LISTING (36 sequences)

SEQ ID NO: 1 - DNA, 288 bp, Artificial Sequence
  Description of Artificial SequenceAluJ consensus sequence
ggccgggcgcggtggctcacgcctgtaatcccagcactttgggaggccgaggcgggaggatcacttgagcccaggagttcgagaccagcctgggcaacatagtgaaaccccgtctctacaaaaaatacaaaaattagccgggcgtggtggcgcgcgcctgtagtcccagctactcgggaggctgaggcaggaggatcgcttgagcccgggaggtcgaggctgcactgagccgtgatcgcgccactgcactccagcctgcgcgacagagcgagaccctgtctcaaaaaa

SEQ ID NO: 2 - DNA, 288 bp, Artificial Sequence
  Description of Artificial SequenceAluSx consensus sequence
ggccgggcgcggtggctcacgcctgtaatcccagcactttgggaggccgaggcgggcggatcacctgaggtcaggagttcgagaccagcctggccaacatggtgaaaccccgtctctactaaaaatacaaaaattagccgggcgtggtggcgcgcgcctgtaatcccagctactcgggaggctgaggcaggagaatcgcttgaacccgggaggcggaggttgcactgagccgagatcgcgccactgcactccagcctgcgcgacagagcgagactccgtctcaaaaaa

SEQ ID NO: 3 - DNA, 289 bp, Artificial Sequence
  Description of Artificial SequenceAluSq consensus sequence
ggccgggcgcggtggctcacgcctgtaatcccagcactttgggaggccgaggcgggtggatcacctgaggtcaggagttcgagaccagcctggccaacatggtgaaaccccgtctctactaaaaatacaaaaattagccgggcgtggtggcgggcgcctgtaatcccagctactcgggaggctgaggcaggagaatcgcttgaacccgggaggcggaggttgcactgagccgagatcgcgccactgcactccagcctgcgcaacaagagcgaaactccgtctcaaaaaa

SEQ ID NO: 4 - DNA, 289 bp, Artificial Sequence
  Description of Artificial SequenceAluSp consensus sequence
ggccgggcgcggtggctcacgcctgtaatcccagcactttgggaggccgaggcgggcggatcacctgaggtcgggagttcgagaccagcctgaccaacatggagaaaccccgtctctactaaaaatacaaaaattagccgggcgtggtggcgcatgcctgtaatcccagctactcgggaggctgaggcaggagaatcgcttgaacccgggaggcggaggttgcgctgagccgagatcgcgccattgcactccagcctgcgcaacaagagcgaaactccgtctcaaaaaa

SEQ ID NO: 5 - DNA, 285 bp, Artificial Sequence
  Description of Artificial SequenceAluSc consensus sequence
ggccgggcgcggtggctcacgcctgtaatcccagcactttgggaggccgaggcgggcggatcacgaggtcaagagatcgagaccatcctggccaacatggtgaaaccccgtctctactaaaaatacaaaaattagctgggcgtggtggcgcgcgcctgtagtcccagctactcgggaggctgaggcaggagaatcgcttgaacccgggaggcggaggttgcactgagccgagatcgcgccactgcactccagcctgccgacagagcgagactccgtctcaaaaaa

SEQ ID NO: 6 - DNA, 287 bp, Artificial Sequence
  Description of Artificial SequenceAluY consensus sequence
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgtggtggcgggcgcctgtagtcccagctactcgggaggctgaggcaggagaatggcgtgaacccgggaggcggagcttgcactgagccgagatcgcgccactgcactccagcctgcgcgacagagcgagactccgtctcaaaaaa

SEQ ID NO: 7 - DNA, 287 bp, Artificial Sequence
  Description of Artificial SequenceAluYa5 consensus sequence
ggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcactgagccgagatcccgccactgcactccagcctgcgcgacagagcgagactccgtctcaaaaaa

SEQ ID NO: 8 - DNA, 286 bp, Artificial Sequence
  Description of Artificial SequenceAluYa8 consensus sequence
ggccgggcgcggtggctcacgcctgtaatcccagcactttgggaggccgaggcgggcggatcacgaggtcaggagatcgagaccatcccggctaaaacggtgaaaccccgtctctactaaaactacaaaaaatagccgggcgtagtggcgggcgcctgtagtcctagctacttgggaggctgaggcaggagaatggcgtgaacccgggaggcggagcttgcactgagccgagatcccgccactgcactccagcctgcgcgacagagcgagactccgtctcaaaaaa

SEQ ID NO: 9 - DNA, 294 bp, Artificial Sequence
  Description of Artificial SequenceAluYb8 consensus sequence
ggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccgggaagcggagcttgcactgagccgagattgcgccactgcagtccgcagtccggcctgcgcgacagagcgagactccgtctcaaaaaa

SEQ ID NO: 10 - DNA, 578 bp, Bos sp.
  normal cow “180” clone # 180T7REV
gagctcggatccactagataacrgccrccagtgtgctggaattcgcccttmagcagtggtaacaacgcagagtactttttttttttttttttttttttttggacatttyattaktatcargactkttraggaacaagatgacactgacatgggttnatcatccccmcagttagaaagtactaagctttatctgtttccttctcaggccaatctctacctctgcattagatatgaagacatctttcttcccattcctaccaraaggaacatttaagactatttaamatgcattgcttctgttggttttacaagtattggctaggcactatktaacggcgaactttagagagggaaaragtggcagttactattggcaaattatcaacctatgtgcagaatccctgctgaatcatttamatattrtcwcatacttatccccgcgtactctgcgttgttcaccmctgctttaaggggcaaawttctacnnatnycccccccccgggggnccgcncngnncntnmatcggggggccmaattsccccctnnngnragbtcntanaacaannccgcggsgscgg

SEQ ID NO: 11 - DNA, 363 bp, Bos sp.
  normal cow “180” clone # 180T9REV
tctcngatcactanttcggccgycantttgctngawttcgcccttmagcagtggtaacaacgyagagtayttttttttttttttttttttnttttwhcaatyattggatythcgnragwtgtttcgtcagaattatcaccattgtyatattcaaaacyaataakkttaaywaagtaattgtyacttacagcgncaattkattcttttgcawctaaattgttttcaatkattacwtcttttatttthttcattwtttcgtwccctttattcmtacctttdatttytttwwggtcntccatcttttangcccytnntckgtntaaaggttctagnggatcttcgtaaattttttgtttttttttt

SEQ ID NO: 12 - DNA, 645 bp, Bos sp.
  normal cow “180” clone # 180T12REV
gagctcggatccactantaacggccgccagtgtgctggaattcgcccttaagcagtggtaacaacgcagagtactttttttttttttttttttttttttttcaatcattggttcttcgtgaratttttcgtcaaaattatcaccattgtmatattcaaaaccaataaggttaacaaagtaattktcacttacagcgtcaatagatacttttgcaactaaatggtttacaatgataacatctgtaattttattcatgatatcgtaccatttattcatacctttgatttctttaaggtcatccatcgttaaagcacctaaacgatataaaggttcagtgaacttcgcaaattccatgaaatttgtatcttctggattgaatgttgatacaaatktttcgttcagtggaatttttccnncttnyaanggncnngannaaaaagnttngccttcaataccatcaattncaaaaaaaagncanacngactgtataaagcmcggaccactgcgnnntanaacnngaaaangttttttyctaagtttcatcmcagtcaanatnnancgaaaggtggtttngncannntcgyvsgaanngggggcccngnnannnnacccnamncnnngnnnngaaanaaaaamccamcccgg

SEQ ID NO: 13 - DNA, 374 bp, Bos sp.
  normal cow “180” clone # 180T18REV
cggatccactagtaacggccgccagtgtgctggaattcgcccttaagcagtggtaacaacgcagagtacgcgggaaaaaaaaaaaaaaaaaaaavaaaaaaaaaaaaaaaaaaaaaaaaaaaatwaaaaaawwaaaaraattrtgagccgcattantnnnnntttntnnnntatwtnnttnnnannnnnntnnttttttttnaaaattnttcyccccbttnntcmcytntggggtkgggggttttcctcccttttcccctttttttnggggcttttnntnnnnnnnnnnntgggcsgcykcccaggggcsscgcggttnttntnnttccannnnnggggggtnttgnnnatttttgtgctbttnnnnnntnncc

SEQ ID NO: 14 - DNA, 801 bp, Bos sp.
  normal cow “180” clone # 180T19REV
cggatccactagtaacggccgccagtgtgctggaattcgcccttmagcagtggtaacaacgcagagtwctttttttttttttttttttttttttttttatycacttctccaaaaagaagaatgattaamtttatttagaataataaacaaatgaaattgatctttacttagatctcggccaattcacggagacgamgaacagtggaacggacartggcagcarcagtagtggagacggtgtaarcaccaccagccatggtcttcttacaacccttacacttccagataccaacagcggtacsttgnacagcatccttwccacagaaggtacaagtgtaggtggaatkttgagtgatttccatcatcttgacagtctttctgggagaggcaccataacggagtaccgtatttaccggtgattccscccttcttaktkcgctttgccattttagattaaataccaaagaggagccccgcgtactctgcgttgttaccactgcttaaggggcgaaattctacagatttmcttcacnctgggggcgctcgnagcntacatatagngggcaccatttccccctatnnannntcantatnacnnnncnnnngggcgggcggkgggkccagcgtcatnnctagganncccctgggcrttncccnncwttnttcgcctttggagannnntccccctttttagccnnnttcggcgttaattarccgagnggaggccmscccccgctcgcccctcamcacacngttacgcgccatnnttggagcsgnatgccgcgcgcccwttnggcgcgc

SEQ ID NO: 15 - DNA, 801 bp, Bos sp.
  normal cow “180” clone # 180T21REV
gttcnggccgnctnccgttggtggctntatgnttccatstgsgtccctgctnttnccgccagtgctctccgtgtttggctntcccncncgctctgbacaaaasaaagccaaacatcaaaaggccggcnntgcntaaagccaacnatanagccctctctcntccgtcnncgccgtnccnngcnntgcggcnngcckcngcggngnnnnctctgcggnntcntgcgggnngnctnttttttctatgtgcccncgcctctngccagcagtntntnttnctcnnttctggcctctttcttgcnnttttctttgtggtcnctttncckgccnttnncccttntcktncncnctcttccttcttctctctctctcttctctttctctctckcttcgcntcntnntntntnntntnnncgncgccttgctttgntttnttttgtcggnttnctgtgtntttntgttntggggntttgntntcctgcnntcscntttccncttttgtgggncttttggcngttttgttnnyncngtgtgtntccntgggcnttgnnncgttnnnntgttsnyctntctcnskttnynnynnynynnnctttntctnttnnnctttgctntcgctgtsngnctgctstngttsntgncgctctntttctttgtnnttnsnttttctctnctctctcttctcnctcgtcgngnsnsnnnctctctctctnygtgtgnnycctgtgtncckctctgtctgntntttcgtckcncnntttttnccgctntgtnntntttnnccyctctcntctnntnttgtnn

SEQ ID NO: 16 - DNA, 590 bp, Bos sp.
  normal cow “180” clone # 180T22REV
gtwaaggcgamtaccgatggwggmtntatgattccatgagggctacctgacaaatnccgcmgatgactcaaagaanataaaannnaanannnnaagkabatttttcgarcggcctrtccnttggccttgacnatataatnggacagaaaataaymmmyctagaaamatmcmcacnagatagcctwtganttttcgccasmmycccccyacgcgaaracgcanngcgggcccgaccggcgaganggcatccggakrggmcccaccatgcangggggtatgccckgaggggntnacacgctgggacantttggccggaaggncctggggggaatgaagaaaacaacngcaaagmatgggccaggaaagrganggannggmgantcccaarcnaaagatwwccgcatarntgantttaannnttccmgaaaaacacccccnanaamcctccaaccaaccgatcnnaaggcaagcangaagggrcgatngccggggggtrtacgaggtgtnnnnccgcgatgaacctatttayccggakktaacsggncgsgcaatttttggkaagntctccaaaamaaacaatttggtccaag

SEQ ID NO: 17 - DNA, 504 bp, Bos sp.
  normal cow “180” clone # 180T28REV
gagctcggatccactagtaacggccgccagtgtgctggaattcgcccttaagcagtggtaacaacgcagagtacttttkttwtttttttttttaantttcaaagtaaaggtacctggttccctgctgccggaacaacagatcaccaagaggaaagacccagcaagacagwactcgccgtgaggcanccgcctgtccaggtcmaaggtccaamwamgagctttttaactgcaacaactttaatatacgctattggagctggaatwaccgcggctgctggcaccagacttgcccnccaatwgttcctcgttaagggatttaaattgtactcattccaattatacgacgnawaaggscgnatattgttacaaattgtcactacctccccgngnnggcgannggggaannngcgcgccnnnngnmttcgtnggangnggnagccanntcncaggnnccnnnccggaatngaancgntanncccgnnaanncgnnnnaannanggnngg

SEQ ID NO: 18 - DNA, 293 bp, Bos sp.
  normal cow “180” clone # 180T29REV
gagctcggatccactagtaacggccgccagtgtgctggaattcgcccttaagcagtggtaacaacgcagagtagcgggactggaagttgagagtacaatgtctacaggaaggaattcatgcatggtatgctgcagtgtgttgagtgcgttgttgtaggggttaccgcgtgtgtggcaattgtgatgatgttggcttgtagcaaatgaccaggagttgatgggtgttggtgattgtwagacaagcatggtgctggtcgattagtatttgtaatagctgtgtctaaaaaaaaaaa

SEQ ID NO: 19 - DNA, 410 bp, Bos sp.
  normal cow “180” clone # 180T30REV
cggatccactagtaacggccgccagtgtgctggnaattsgcccttdagcagtggtaacarcgcagagtacgcgggtkatrrragaccaaatrrdatataadrdgcagtaaaadrdrrrrdddkratkrdnrgrkacataaagataaadkgrggtgaaatrraagggggntnggggbkkttkkggnknnttcngaggttnngrcaraagttratgggcagaaaaggkgtgntntgncnktnntgbktkgggccnnncscbtcnnnnncctnnnncttgngngbkggnknnscggnnkscngnttcccnnnnkbkkkktkkbgcknantcnntnnnacnnccgngbgggngngnggkgnggkcnncccccnccnnnnccnnncccgggsggnggtgntnngattgntngsgg

SEQ ID NO: 20 - DNA, 798 bp, Bos sp.
  normal cow “180” clone # 180T31REV
agctcggatccactagtaacggccgccagtgtgctggaattcgcccttaagcagtggtaacaacgcagagtacgcgggccagtgatatattcagatcctattccttcaatgagatttccaaccattcagtttcaatatgttcaaaaaagaagattcaagtaatcatgcacatcaaacacttttttcactgatcacctctttttttttattcgcaaatcaaatcatcactttttgttattttccaaaggagcattttttttctcctatattttactaggttaattttagtatatattatttatkatttatkatkatkatcatctgakttggtggttctcatggctggtctggttggtgcttacctggtgatctcttgggttctgttcctctggtcttttgatctatgtcaagtgtaattgggcnccttttttgcttactggtcttttgatactcgcgagttagccctaggcttttctttttttcggcagaatgtatacatttgatgatgagatttattttngaggantnattctattcagctcactttctacaacttcagcttcaaacattttnagncgangggacannggngnannnaaattnngnaananccanaancncaccantntgkrtktnnggagratcnctnnaagagcnntnckbtntngnnnnngaaggggaagncttnnanggggggantcnatgcnnnnnnggggnnnaraaagttnnganntncgttncgttaaatggnnnnnannnggnngcnantggggnnnngnnnggncaaacaaaacnnnc

SEQ ID NO: 21 - DNA, 239 bp, Bos sp.
  BSE cow “200” clone # 200T4REV
tcgnntytgantccnctatatnyagcyaccagtktnmtngnnttcagcmctwtngmtatrgttnytaaganttatttcgkgnntgktttttnctctnattcttngagaanngtntnnnthtatcnctnctggtthtnancnnayttgncnctgttnnkgnttntcccynthtttnttttttcattnngnctcntttncctnnnntgnancnggtngnnnccntcnnnntttnggtnncc

SEQ ID NO: 22 - DNA, 741 bp, Bos sp.
  BSE cow “200” clone # 200T5UNI
tagggcgaattgggccctctagatgcatgctcgagcggccgccagbgtgatggatatctgcagaattcgccsttaagcagtggtaacaacgcagagtacgcgggacagttcckttggaattatagcatagaaatcgacttcaaaatggctcaacgtgttacttacagaagaagaaatccatgtatgtttatatctaagtatgaccaggactattgaactataagaatgaaagagaagagcaaaggacggtgatacggtccaatgagaaatttatgacaaaatacaataccaaatctaacaagattaaggttgttaagaccscaggtggtaaattagttgcccaacacgtcaagaagctcgcttctagaccaaagtgtggtgactgtggtgatgstttacaaggtatctbtactttaagaccaagagaatacgctcaagtttctaagacccacaagaccgtccaaagagcstacggtggttstagatgtgctaactgtgtcaaggaaagaattgtcagagctttstctgatccgagnacaaaagatcgttaagggagtgttgaaagaacaacaagataaggcacaaaagtccgcccaaggangaccggkaagaaatangttaacttaggnttgamgctttgttatatctagtttttggttttratggttcttctatgtaaatttctttgtcgttttaatacacatttttacgttacaaaacggcgcgcncnnnnnnnctsgc

SEQ ID NO: 23 - DNA, 474 bp, Bos sp.
  BSE cow “200” clone # 200T6REV
cratccctagataayrgccagccantttgctngaattcgcccttaakmwatagttacwamgcagaatacgctgggggakgyttaatttgctgatagaaamaagatcatgccattacactccancctggagcaacaaagagctaaattccttcttaaataaataaatagccagatagcggtgagctcacacctwgttatcccancnctttggtaggcaaaggctatttggttcwyatttgtcanratttctakaccaagcctggccancatagtgaaacmmaaatcatcwtamataaaatattctannttttaaccactgcgtagtgatngcatttgcactgtaatcccagctaacgtgggaggcangggantcacctgnagccggakccggangttttcaagtaagtckngatcagccamtgmactccaammtgkggkncaaaacaatamtcagtctcgnnnnnknnnnttttt

SEQ ID NO: 24 - DNA, 589 bp, Bos sp.
  BSE cow “200” clone # 200T7REV
gaatccactanataacaggccgccagtgtgmtggaattcccccttaanmaatagtaacaamgcagaawcgcggggataaggaaatacacaaagaaawawraaaammaaaacaaagggataaaamtaccgttttagatgaactttttcagcaaacttggtattacagatagggaatgccaaagagaggctagattctatttattcatcttcatatagctgggctaaagggcatagcaaaggattttaatttaggtcatatgatttaggggttngcattttttacggtacataaatttggggtctgtrmgcatgwgttngctanagaggatctaagcaggctattcagnnntcattnnngnctnnagaacaaaagaaggscaggtgtaatggctcatgcctataatcccagcactttagagaggccgaggtrgaaggatttntttagggcaggntttcaagnncagcctggncnnaccgcngnccttttctctgcaaaaatttttatcnattagccanttntggtggtncntncctgttcttngctaccttaaagngggstgnnnnnngganaattcnncctnngggttna

SEQ ID NO: 25 - DNA, 480 bp, Bos sp.
  BSE cow “200” clone # 200T8REV
ggatccactagtaacgkccgccagtgtgctggaattckcccttaagcagtggtaacaacgcagagtacttttttttttttttttttttttttttttaaamaatttttttwtwttgagaraaswcacavacwctgagkggytyyccttcwaaacagctcamaamccaaaaawstgcytccccatttkttswcattgcywyatcragggtagcrgggggttkwttttttggctccccccacccccatctmaaraagagaaaagggggycctkkttttwyttcmcagagtgtckrgrrggttcccccycctstcattwttactrwmaaaaatcgttctgwcacagtgatttsycyttcccccgcgactctgcgttttymccactkttwaggggmrawttctgaagatwtcyatcacactgggggccgctagagcatacatctagggggcccaatncncnnnnnngngagtggtwtcaagkbkt

SEQ ID NO: 26 - DNA, 801 bp, Bos sp.
  BSE cow “200” clone # 200T9REV
gatccactaktaacggccgccagtgtgctggaattytcccttaagcagtggtaacaacgcagagtacgcgggctaagaactcgaatgatgcntaakctatccaacccactttcctcagtttttatttacgcagaaattgaaattaacaaactaatttctgagtcttaagtgaaattgttttcgttttatttaaagttaccnttgacttacatagttttaaccttctggaaagttcttcttttttctctctctcttcctgttcctgattgccacactggatagaaagttctaaatgaatacttaggtttgaaagtatttttctacatctatttattttgccattstacagatttggctccattgtcctaaggtttcagcacaataaaaatctcgtttctcccagtgcttgtsttgacatcaatgtagcattaaaaagtccaagacttttacagactaatttaagctgaacaaggaaatataatctggtacctactaagaaacataaatgggcctgggttgctagcagttcctggaagtcactgtgttttccaataaatccatgcaatgcgtaacagggaaagagaagccacaaagcagaggctgggactgttgggatcatgttagtaattaaactctctctcctcttgagactcagtgagagccttcccttacaaagaaaaaagtgacacagttgctgccaaagcggatgtgacacgcgtcctctcactctaagagattaaaataaangcctcbtgcagtccttttctccaaatacacnaaanaanttggctgggctgaagg

SEQ ID NO: 27 - DNA, 354 bp, Bos sp.
  BSE cow “200” clone # 200T10REV
ccccdabncgsccgccantgkscgggnntkmacbnnbsggcgggsskgcggcgcngagktcgggggggggggttgggggtggtggaaanaaatttttwttatnnnnnnnttnawmttttttttttnnnntnttnttttttttttttntttttnnktntttttttnntnntnttttttttnntnttnnttggntncnggnnnttttnnnnttnttgttgggtgsggcgcggsssssscccssgcnaaatannttcggggtggtgggggtgttggtnnnntkggktgtnnnnntttatttgttttnnntnntttkgttgtgtgkgkkgtggtgggggggggggggsggcgggtggg

SEQ ID NO: 28 - DNA, 734 bp, Bos sp.
  BSE cow “200” clone # 200T11REV
tccactagtaacggccgccagtgtgctggaattcgcmnttaagcagtggtaacaacgcagagtacttttttttttttttttttttttttttttttttthttgagtaggagttttgmtatttttgcccaggctggcgtgcaatggtatgatctcggttmaccgaaamnttcgttttytggattcaggctagtcmattgcctmagcctcvvaagtagctgggattamaggsgggcagmacmatgtgtgghtaattttktatttttadwaaaaatggggtttctcmatgttgatmaggvtggthtaaaactcccaacctcaggtgwtccacccgsnttggcctcvaaaattgctgggtttaaaggtgtgagccaaagtgcccggcctctgmattagttttytaagaawaatggtctcaagcttaataaatgttwttgcaaaggaaatgatcattttttaaatggcccacagagtattcyatgacccgcgtantttgggttggtaaaaatgtttaaggcgggaattttnagatataataaaatggggggcntttnnaattaattttagagggsccattaccctataatnantcntnttataattwtattntngggagtgrtstktataaaaatgtannntnggnaaaaaatagangntnaaaaaaataaattnngnktttgtcgnanntnnncnyttttncnnanntaggngknnttnangaagngggnnggnnnccg

SEQ ID NO: 29 - DNA, 801 bp, Bos sp.
  BSE cow “200” clone # 200T12REV
tcggatccactagtaacggccgccagtgtgctggaattcgcccttaagcagtggtaacaacgcagagtacgcgggtgctgtcactgggaacctggacgacttctcacttgaaaacttgcaccacctcatgccttcccttatcataagcctgggcaacctcntaatattctccccctcacaatagagacaaggcaacctgcagtaaaagtttgagcaaactggccgggcgcgatgcctcgtgcctgtaatcccagcgctttgggaggccgaagtgggtggatcacttgaggtcaggaattcgagaccagtctggccaacatggtgaaaccccgtctctactaaaaataccaaaaaaatgagctaggcatggtggcacatgcctgtaatcccagctactcggaaggctgaggcaggagaatctcttgaacccaggaggtggatgttgcggtgagccgagatcctgccattgcactccagcctgggcaacagcacgagactccatcycaaaaaaaaaaaaaannnnnnnnannnnctytccgttgtwaaccactgctgtaagggggcatattttcgtgsngnttttccatcccnntkgggnggncmgttngnnnaattnnatttgtngnaggggccccattttyccccccttttnnntgnantcnntnntaygayattgtbascgngscgcggtggtgkkrttgwkgatnggktnnnttnnncntgnggganaaaacccccgtngngngnnnwaaaccaaaaacangtnnntggccttngnnnnnnnntccncntttttnncnn

SEQ ID NO: 30 - DNA, 801 bp, Bos sp.
  BSE cow “200” clone # 200T13REV
gcggrgccactagtaacggscgccagtgtgctggaatkcgccsttaagcagtggtaacaacgcagagtacttttttttttttttttttttttttttcatttgcttaactggaaaggcgtttccaattgattgaatagcaacgataactcctcbtataatgccagtagctatctttttgtcttgctcgtctattcgatcccagcccgacaaaatggtaattatcaaagtaagaacgatcgtggtaattgttaaccstgcatcccaacttcgattgattcgtctgagccgagttatttcttgagttaattctttaatttctttctctaagagtttcctttgcttctcgattatagataaagattgagtttgcattttttctctgatttttgagctgagacaagttttgagtaaatgcaccttgctgtgagaaacagtcagtctgatatctacttaagcaggtaacgctttttcataagccttagcaagtttgatgtggnattcattctgccaatacagctctccgttatagccaaaagcgaacttatcccaatctttaagtttgagatgaactagcaaatttctgttcacaataaagttcatcatgtgtcttgcttgccaatactctcctgcaaagttttctcaatgaactgctggattgaatgccacaccagcagcagtntaaktgaatccccatgacttgnttcgagtccaaaagatgcactttttatagcctgaaattcatcaaaatkccatttggmatcaagtaaacgttttatcccactcytgctgacccccccaaaa

SEQ ID NO: 31 - DNA, 577 bp, Bos sp.
  BSE coe “200” clone # 200T14REV
agctcggatccactagtaacggccgccagtgtgctggaattcgccmttaagcagtggtaacaacgcagagtacttttttttttttttttttttttttttntaaacvwawaaatktgtatwaaaaacgacaaaraaatttacatagaagatmccattaaaaaccaaaaactagatattacaaagcttcaatccttaagttaacttatttcttaccggtcttcttggcggacttttgtcccwtatcttgttgttctttcaacaccccnhtaacgatcttttkttcttcgawaaaaaaagctctgacaattcttyccttgacacagttagcacatctagaacccccgtagcyctttggacggccttnggggncttaraaacttgagcgnattcccttggtcttaaagtaganaaaccttgtaaagcatcaccacagncaccacacnttggtctaaaancgaccttcttacgttttgggmcaactaatttaccacmskgggggtctttaacgcccgtgnccttgnanantgggaawgaatnnngccaaaaaakncncatttggaccgnatcaccgnccttt

SEQ ID NO: 32 - DNA, 648 bp, Bos sp.
  BSE cow “200” clone # 200T15REV
gagctcggatccactagtaacggccgccagtgtgctggaattcgcccttaagcagtggtaacaacgcagagtacgcgggatacttaagttctgttaaagttagcaactactttcgtattaaatcatcaagaaaatcacataggccstaawwattataaaagaaaatatkctataaaaaggaatgaaatgntgccatttgcagagacctggatagacctagagactgtcataagactgaattagtcagaaagagaaaaacaaatatcatttgttatattattaacacataatgtggaatctagaaaaatggtatagattatctttttgcaacacagnngttgagacaccgatgtagagaacatctggatgctggkgggggaaggagaaagggggtgatgaattgggagattgggattgatatntntnccctnctngctgtaagtngntaactaatgngnacstgctgtncngcncnnggnnntttactcagtgctctatggtgacctcnnntggnnnngnnntcbnnnckgagaggggatatatgtaaacatatcgttgnttccctttggctntngggccgnnactnncccnattttgttnngccccttttttccnntnnvngntnntttaaatgcgggnnnngaggcg

SEQ ID NO: 33 - DNA, 72 bp, Bos sp.
  Alu-like sequence containing fragment from BSE cow
aagatcatgccattacactccancctggagcaacaaagagctaaattccttcttaaataaataaatagccag

SEQ ID NO: 34 - DNA, 70 bp, Homo sapiens
  human sequence containing Alu repetitive sequences
aagatcatgccattgcactccagcctgggcaacaagagctaaattccttcttaaataaataaatagccag

SEQ ID NO: 35 - DNA, 111 bp, Bos sp.
  Alu-like sequence containing fragment from BSE cow
gcgtagtgatngcatttgcactgtaatcccagctaacgtgggaggcangggantcacctgnagccggakccggangttttcaagtaagtckngatcagccamtgmactcca

SEQ ID NO: 36 - DNA, 108 bp, Homo sapiens
  human sequence containing Alu repetitive sequences
gcgtggtgatggcatatgcctgtaatcccagctacgtgggaggcaggggaatcacttgaagccggaagccggaggttgcagtaagtcgagatcagccactgcactcca

(19) United States
(12) Patent Application Publication
(10) Pub. No.: US 2010/0261164 A1
(43) Pub. Date: Oct. 14, 2010

(54) Diagnostic detection of nucleic acids

(75) Inventors: Ekkehard Schuetz, Goettingen (DE); Howard B. Urnovitz, Iowa City, IA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Chronix Biomedical, Inc., San Jose, CA (US)

(21) Appl. No.: 11/803,650

(22) Filed: May 14, 2007

Related U.S. Application Data
(63) Continuation of application No. 10/762,966, filed on Jan. 21, 2004, now abandoned.
Application No. 10/762,966 is a continuation of application No. 10/115,278, filed on Apr. 1, 2002, now abandoned.
(60) Provisional application No. 60/280,523, filed on Mar. 30, 2001.

(51) Int. Cl. C12Q 1/68

(52) U.S. Cl. 435/6

(57) ABSTRACT

This invention provides sensitive nucleic acid hybridization assay methods for the detection of target animal nucleic acids in a biological sample, such as acellular fluids. The methods are particularly useful in early diagnosis of animal diseases, particularly chronic illnesses.

### CROSS REFERENCE TO RELATED APPLICATIONS

This application is a continuation of U.S. application Ser. No. 10/762,966 filed Jan. 21, 2004, which is a continuation of U.S. application Ser. No. 10/115,278 filed Apr. 1, 2002, which claims benefit of U.S. Provisional Application No. 60/280,523 filed Mar. 30, 2001, all of which applications are herein incorporated by reference.

### FIELD OF THE INVENTION

The invention relates to the detection of nucleic acids associated with animal disease states. In particular, the invention provides for the detection of nucleic acids, particularly RNA, in acellular biological fluids as diagnostic assays for chronic illnesses and infectious diseases. Also provided are therapeutic approaches to treating diseases in animals.

### BACKGROUND OF THE INVENTION

It is known that environmental and other factors (e.g., genotoxic compounds, infectious retroviruses, retroelements and the like) can directly and indirectly disrupt and/or damage DNA and may play a role in the development of a number of illnesses in animals. The mechanisms by which damage to genetic material leads to the onset of these diseases is not well understood, however. It is known that certain sites in the genome are especially susceptible to such modifications, for example, the distribution of insertion sites for retroviruses and retroelements is not random as fragile sites are often preferred (see, e.g., Craigie Trends in Genetics 8:187 (June 1992); De Ambrosis et al. Cancer Genet. Cytogenet. 60:1-7 (1992); Durnam et al. and Romani et al. Gene 135:153-160 (1993)). Moreover, repetitive sequences, including tandemly repeated DNA and interspersed repetitive DNA that is present throughout the genome have been implicated in many diseases and are often involved in chromosomal alterations, e.g., mutations and rearrangements such as deletions, duplications, and abnormal recombinations, that result from DNA damage (see, e.g., Purandare & Patel, Genome Res. 7:773-786, 1997 for review of repetitive sequences and involvement in human disease).

Detection and confirmation of nucleic acids from pathogens such as bacteria, parasites and viruses, is a commonly used method for diagnosis of disease in animals. For instance, detection of viral, bacterial, or other sequences is useful in diagnostic procedures. The present invention provides additional methods for risk assessment and the diagnosis and treatment of disease. These methods detect the presence of nucleic acid molecules, typically RNA molecules, in acellular fluids of animals. Often, these nucleic acid molecules comprise repetitive sequences or fragments thereof or fragments thereof recombined with other nucleic acid material transcribed from the animal genome. In one embodiment, the invention provides methods of detecting an RNA molecule containing repetitive sequences transcribed from animal germline DNA in an acellular sample obtained from an animal with a particular disease. The presence of the RNA molecule or sentinel gene sequences indicates the presence of the disease.

### SUMMARY OF THE INVENTION

The present invention provides methods of screening for a disease state in an animal. The methods comprise providing a sample containing biological material (e.g., biopsies) or biological fluids from the animal (e.g., an acellular biological fluid such as serum or plasma) and contacting the sample with a nucleic acid which specifically hybridizes to a target nucleic acid sequence. The target nucleic acid sequence is typically RNA. Often, the method of screening comprises a step of detecting at least a second target nucleic acid sequence to detect the presence of a disease state. In some embodiments, dection of the target RNA comprises detecting a biological activity of the target RNA.

In some embodiments, the target nucleic acid includes sequences from the genome of the animal that are transcribed from repetitive sequence elements and/or chromosomal rearrangements. In some embodiments, the sequences are from a fragile site in a genome. In other embodiments, the target nucleic acid may be a novel rearrangement of animal germline sequence or a novel composite of animal germline sequences and sequences of microbial or viral origin. The target nucleic acid is usually at least about 100 nucleotides in length, sometimes between about 500 and about 1500 nucleotides in length. Fragments detected in rearranged sequences are typically at least about 40 nucleotides in length.

The methods are often used to detect specific diseases, often chronic illnesses such as cancer and chronic wasting diseases, and spongiform encephalopathies such as bovine spongiform encephalopathy (BSE), foot-and-mouth disease, porcine reproductive and respiratory syndrome, swine vesicular disease and others.

In certain preferred embodiments, the target animal nucleic acids are amplified (e.g., by PCR).

The present invention further provides improved methods for detecting RNA molecules that comprise sequences transcribed from repetitive germline DNA of animals such as cows, deer, swine, sheep, mink, elk, and goats, in biological samples and vaccine preparations.

In one embodiment, the invention provides methods for detecting recombinant animal-viral nucleic acids, which may comprise nucleic acid sequences from a first virus and a second virus. Often, the sequence of one of the viruses, or fragments thereof, are harbored in the genome of the animal. The sequence may be in germline DNA or may be acquired post-infection. The methods comprise contacting a biological sample suspected of containing the recombinant viral nucleic acid with a first primer which specifically hybridizes to a conserved or sentinel sequence in a first viral genome and a second primer which specifically hybridizes to a nucleic acid sequence from a second virus. The presence of an amplified product which is a recombinant animal-viral nucleic acid is then detected.

A number of primers may be used in the present invention. For instance, one or both the primers may specifically hybridize to a 5′ nontranslated region of a viral genome. Since the 5′ nontranslated region is conserved among picornaviruses, the primer will specifically hybridize to most picornaviruses, particularly enteroviruses. However, when an animal is exposed to a genotoxic event, recombinatorial events may create novel sequences of animal origin that hybridize with enteroviruses primers.

The methods may be carried out using a number of biological samples commonly used for clinical analysis of nucleic acids. A convenient sample is serum or plasma.

A number of methods may be used to detect the presence of the recombinant, i.e., rearranged nucleic acid. In some embodiments, the detection is carried out using gel electrophoresis to identify an amplified fragment that is not present in a control sample known to contain only virus nucleic acids.

In some embodiments, populations of recombinant nucleic acid can be detected using arrays of gene or other nucleic acid sequences.

The invention further provides nucleic acid molecules from new, recombinant sequences identified here. The claimed molecules can be identified by their ability to hybridize to the exemplified sequences under stringent conditions, as defined below. The nucleic acids may be a complete animal gene or fragments thereof, or may be a sequence corresponding to a nontranscribed region of the animal genome. The nucleic acids may be isolated from a biological sample and may or may not be integrated in animal chromosomal DNA.

### BRIEF DESCRIPTION OF THE DRAWINGS

FIG. 1 shows Alu consensus sequences (SEQ ID NOs. 1-9) as shown in Batzer et al., J Mol. Evol 42:3-6, 1996.

### DEFINITIONS

An “acellular biological fluid” is a biological fluid which substantially lacks cells. Typically, such fluids are fluids prepared by removal of cells from a biological fluid that normally contains cells (e.g., whole blood). Exemplary processed acellular biological fluids include processed blood (serum and plasma), urine, milk, saliva, sweat, tears, phlegm, cerebrospinal fluid, semen, feces and the like.

An “archived nucleic acid sequence” is a chimeric sequence in animal genomic DNA containing subsequences from other organisms, particularly pathogens such as bacteria (e.g., members of the genera Chlamydia, Mycoplasma, Neisseria, Treponema, Staphylococcus, Streptococcus, and the like), parasites (e.g., Plasmodium falciparum, Pneumocystis carinii, Trichomonas, Cryptosporidium), viruses (e.g., herpes viruses, enteroviruses, polyoma viruses, poxviruses, such as Molluscum contagiosum viruses, retroviruses, and the like). Thus, when designing nucleic acids (e.g., as probes or PCR primers) for detecting archived nucleic acids of the invention, sequences based on the genome of these pathogens are conveniently used. Without wishing to be bound by theory, it is believed that archived nucleic acid sequences are usually inserted at repetitive sites or recombination hotspots.

“Nucleic acid” refers to a deoxyribonucleotide or ribonucleotide polymer in either single- or double-stranded form, and unless otherwise limited, would encompass known analogs of natural nucleotides that can function in a similar manner as naturally occurring nucleotides.

The term “biological sample”, as used herein, refers to a sample obtained from an organism or from components (e.g., cells) of an organism. The sample may be of any biological tissue or fluid. Frequently the sample will be a “clinical sample” which is a sample derived from an animal with a disease or suspected of having a disease (a “patient”). Such samples include, but are not limited to, sputum, blood, serum, plasma, blood cells (e.g., white cells), tissue or fine needle biopsy samples, urine, milk, peritoneal fluid, and pleural fluid, or cells therefrom. Biological samples may also include sections of tissues such as frozen sections taken for histological purposes.

An “animal” as used herein refers non-human animals, often mammals, including, but not limited to primates such as chimpanzees and monkeys, horses, cows, deer, sheep, goats, pigs, dogs, minks, elk, cats, lagromorphs, and rodents.

A “chronic illness” is a disease, symptom, or syndrome that last for months to years. Examples of chronic illnesses in animals include, but are not limited to, cancers and wasting diseases as well as autoimmune diseases, and neurodegenerative diseases such as spongiform encephalopathies and others.

“Repetitive sequences” refer to highly repeated DNA elements present in the animal genome. These sequences are usually categorized in sequence families and are broadly classified as tandemly repeated DNA or interspersed repetitive DNA (see, e.g., Jelinek and Schmid, Ann. Rev. Biochem. 51:831-844, 1982; Hardman, Biochem J. 234:1-11, 1986; and Vogt, Hum. Genet. 84:301-306, 1990). Tandemly repeated DNA includes satellite, minisatellite, and microsatellite DNA. Interspersed repetitive DNA includes short interspersed nuclear elements (SINES) and long interspersed nuclear elements (LINES).

A “rearranged sequence” or “recombined sequence” is a region of the genomic DNA that is rearranged compared to normal, i.e., the rearranged sequence is not contiguous in genomic DNA in healthy animals or in genomic DNA obtained from animals prior to contracting a disease or prior to exposure to a genotoxic agent.

A “fragile site” is a locus within an animal genome that is a frequent site of DNA strand breakage. Fragile sites are typically identified cytogenetically as gaps or discontinuities as a result of poor staining. Fragile sites are classified as common or rare and further divided according to the agents used to induce them. For a general description of fragile sites and their classification, see, Shiraishi et al PNAS 98 :5722-7 (2001), Sutherland GATA 8:1961-166 (1991). Exemplified sequences disclosed herein include sequences that are found in rearrangments of host genomic DNA or viral genomes that have apparently been inserted into the animal genome at a fragile site. Thus, fragile sites can contain “archived nucleic acid sequences” that are from the host and/or pathogens, including bacteria, parasites, and viruses.

A “target animal nucleic acid” of the invention is a nucleic acid molecule from animal genomic DNA (e.g., chromosomal DNA, mitochondrial DNA, and other extrachromosomal DNA). As used herein, animal genomic DNA often refers to germline DNA, however, animal genomic DNA may also include nucleic acids introduced into the individual as a result of infection of the individual by a pathogenic microorganism (e.g., exogenous viral DNA integrated into the genome after infection or through live virus infection). Thus, although target animal nucleic acids of the invention are of animal origin, they may nonetheless contain sequences shared by other pathogenic organisms, such as viruses. Such sequences are sometimes referred to here as animal/viral chimeric sequences or “archived sequences”. DNA “derived from” animal genome DNA includes DNA molecules consisting of subsequences of the genomic DNA as well as RNA molecules transcribed from animal genomic DNA.

The RNA molecules detected in the methods of the invention may be free, single or double stranded, molecules or complexed with protein. Such RNA molecules need not be transcribed from a gene, but can be transcribed from any sequence in the chromosomal DNA. Exemplary RNAs include small nuclear RNA (snRNA), mRNA, tRNA, and rRNA. Often, the RNA molecules detected in the acellular sample comprise sequences transcribed from repetitive DNA in the animal genome.

A “sentinel” nucleic acid sequence is a sequence that indicates the presence of a disease or condition in an animal. The sequence is typically present in affected animals and absent in normal animals, although, in other embodiments, the “sentinel” may be present only in normal animals. Alternatively, the relative level of the sequence in a biological sample, e.g., acellular fluid, from an animal may be increased or decreased in comparison to the level in normal animals. A sequence that is “upregulated” in diseased animals is present in increased levels relative to a control. A sequence that is “downregulated” in diseased animals is present in decreased levels relative to a control.

A “profile” refers to a collection of sentinel sequences that are useful for diagnosis. A profile can comprise up-regulated sequences, down-regulated sequences or a combination of both up-regulated and down-regulated sequences.

As used herein, “vironomic nucleic acids” are nucleic acids that comprise a profile of sentinel genes associated with a disease state. As described above, the vironomic nucleic acids disclosed herein are often derived, in part, from the host, although the vironomic nucleic acids of the invention may comprise sequences derived from other microorganisms or from cellular sequences or recombinations therewith.

The terms “hybridize(s) specifically” or “specifically hybridize(s)” refer to complementary hybridization between an oligonucleotide (e.g., a primer or labeled probe) and a target sequence. The term specifically embraces minor mismatches that can be accommodated by reducing the stringency of the hybridization media to achieve the desired priming for the PCR polymerases or detection of hybridization signal.

“Nucleic acid” refers to a deoxyribonucleotide or ribonucleotide polymer in either single- or double-stranded form, and unless otherwise limited, would encompass known analogs of natural nucleotides that can function in a similar manner as naturally occurring nucleotides.

The term “oligonucleotide” refers to a molecule comprised of two or more deoxyribonucleotides or ribonucleotides, such as primers, probes, and other nucleic acid fragments. The exact size of an oligonucleotide depends on many factors and the ultimate function or use of the oligonucleotide. “Adding” an oligonucleotide refers to joining an oligonucleotide to another nucleic acid molecule. Typically, adding the oligonucleotide is performed by ligating the oligonucleotide using a DNA ligase.

The term “primer” refers to an oligonucleotide, whether natural or synthetic, capable of acting as a point of initiation of DNA synthesis under conditions in which synthesis of a primer extension product complementary to a nucleic acid strand is induced, i.e., in the presence of four different nucleoside triphosphates and an agent for polymerization (such as DNA polymerase or reverse transcriptase) in an appropriate buffer and at a suitable temperature. A primer is preferably a single-stranded oligodeoxyribonucleotide sequence. The appropriate length of a primer depends on the intended use of the primer but typically ranges from about 15 to about 30 nucleotides. Short primer molecules generally require cooler temperatures to form sufficiently stable hybrid complexes with the template. A primer need not reflect the exact sequence of the template but must be sufficiently complementary to specifically hybridize with a template.

“Probe” refers to an oligonucleotide which binds through complementary base pairing to a subsequence of a target nucleic acid. It will be understood by one of skill in the art that probes will typically substantially bind target sequences lacking complete complementarity with the probe sequence depending upon the stringency of the hybridization conditions. The probes are typically directly labeled (e.g., with isotopes or fluorescent moieties) or indirectly labeled such as with digoxigenin or biotin. By assaying for the presence or absence of the probe, one can detect the presence or absence of the target.

The term “cis-regulatory sequence” refers to cis-acting sequences (either 5′ or 3′) necessary for efficient transcription of structural sequences (e.g., open reading frames). These sequences include promoters, enhancers and other sequences important for efficient transcription and translation (e.g., polyadenylation sites, mRNA stability controlling sequences and the like). A “regulatory sequence” also refers to a nucleic acid sequence that regulates gene expression, changes in cell surface characteristics, or elicits other phenotypic effect on a population of cells into which it is introduced.

A “sequence specific to” a particular virus species or strain (e.g., an animal virus) is a sequence unique to the species or strain that is not shared by other previously characterized species or strains. A probe or primer containing a sequence complementary to a sequence specific to a virus will typically not hybridize to the corresponding portion of the genome of other viruses under stringent conditions (e.g., washing the solid support in 0.2×SSC, 0.1% SDS at about 60° C., preferably 65° C. and more preferably about 70° C.).

The term “substantially identical” indicates that two or more nucleotide sequences share a majority of their sequence. Generally, this will be at least about 90% of their sequence and preferably about 95% of their sequence. Another indication that sequences are substantially identical is if they hybridize to the same nucleotide sequence under stringent conditions (see, e.g., Sambrook and Russell, eds, Molecular Cloning: A Laboratory Manual, 3rd Ed, vols. 1-3, Cold Spring Harbor Laboratory Press, 2001; and Current Protocols in Molecular Biology, Ausubel, ed. John Wiley & Sons, Inc. New York, 1997). Stringent conditions are sequence-dependent and will be different in different circumstances. Generally, stringent conditions are selected to be about 5° C. (or less) lower than the thermal melting point (Tm) for the specific sequence at a defined ionic strength and pH. The Tₘ, of a DNA duplex is defined as the temperature at which 50% of the nucleotides are paired and corresponds to the midpoint of the spectroscopic hyperchromic absorbance shift during DNA melting. The Tₘ, indicates the transition from double helical to random coil

Typically, stringent conditions will be those in which the salt concentration is about 0.2×SSC at pH 7 and the temperature is at least about 60° C. For example, a nucleic acid of the invention or fragment thereof can be identified in standard filter hybridizations using the nucleic acids disclosed here under stringent conditions, which for purposes of this disclosure, include at least one wash (usually 2) in 0.2×SSC at a temperature of at least about 60° C., usually about 65° C., sometimes 70° C. for 20 minutes, or equivalent conditions. For PCR, an annealing temperature of about 5° C. below Tm, is typical for low stringency amplification, although annealing temperatures may vary between about 32° C. and 72° C., e.g., 40° C., 42° C., 45° C., 52° C., 55° C., 57° C., or 62° C., depending on primer length and nucleotide composition or high stringency PCR amplification, a temperature at, or slightly (up to 5° C.) above, primer Tm is typical, although high stringency annealing temperatures can range from about 50° C. to about 72° C., and are often 72° C., depending on the primer and buffer conditions (Ahsen et al., Clin Chem. 47:1956-61, 2001). Typical cycle conditions for both high and low stringency amplifications include a denaturation phase of 90° C.-95° C. for 30 sec-2 min., an annealing phase lasting 30 sec.-2 min., and an extension phase of about 72° C. for 1-6 min.

As used herein a “viral nucleic acid” is a nucleic acid molecule comprising nucleic acid sequences derived from viruses. Since as described below, the viral nucleic acids disclosed here are thought to be derived from recombination events, the viral nucleic acids of the invention may contain sequences derived from other microorganisms or from cellular sequences.

A nucleic acid comprising a “complete viral genome” is a nucleic acid molecule encoding all the polypeptide products required to construct a complete, infectious viral particle. As used herein a complete, infectious viral particle can be encoded by a sequence that is a full length genome, as well as a substantially full length (e.g., 90%, preferably 95% complete) genome.

The terms “identical” or percent “identity,” in the context of two or more nucleic acids, refer to two or more sequences or subsequences that are the same or have a specified percentage of nucleotides that are the same (i.e., about 70% identity, preferably 75%, 80%, 85%, 90%, or 95% identity over a specified region, when compared and aligned for maximum correspondence over a comparison window, or designated region as measured using a BLAST or BLAST 2.0 sequence comparison algorithms with default parameters described below, or by manual alignment and visual inspection. Such sequences are then said to be “substantially identical.” This definition also refers to the complement of a test sequence. Preferably, the identity exists over a region that is at least about 15, 20 or 25 nucleotides in length, or more preferably over a region that is 50-100 nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 15 to 600, usually about 20 to about 200, more usually about 50 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the default parameters described herein, to determine percent sequence identity for the nucleic acids described herein. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

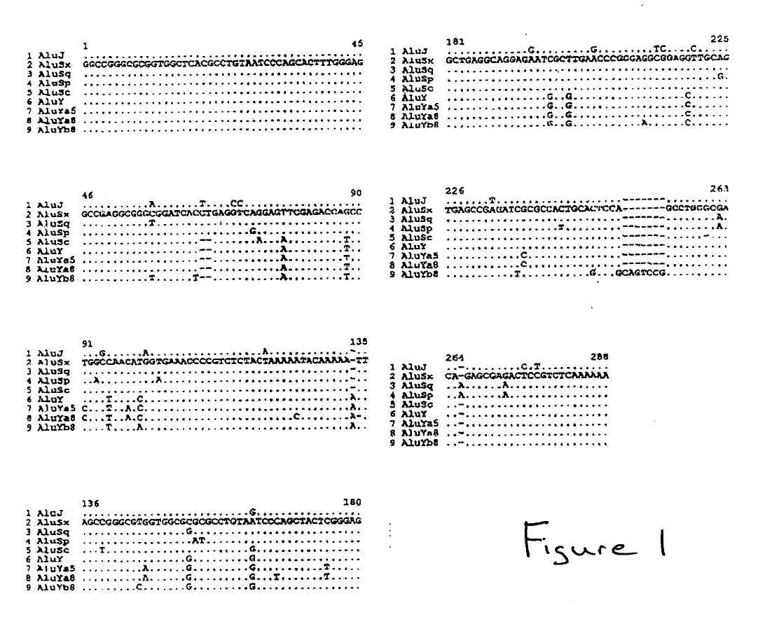

An “Alu-like sequence” as used herein refers to a repetitive element present in a nonprimate animal that has at least 70% sequence identity, often at least 80% or greater sequence identity, to a consensus Alu sequence. Alu sequences comprises a consensus sequence of about 280 base pairs, typically followed by an A-rich region and flanked by direct repeat sequence representing the duplicated insertion site. Alu consensus sequences are known in the art, see, FIG. 1 in Batzer et al., J Mol. Evol 42:3-6, 1996. These sequences are reproduced in FIG. 2 in the current application.

### DETAILED DESCRIPTION OF THE INVENTION

Although human RNA has been detected in acellular biological samples (see, e.g., WO9814617), acellular animal RNA has not been identified. The present invention is based in part on the surprising discovery of novel RNA molecules, including transcripts from archived nucleic acids, in biological fluids. In some embodiments, RNA molecules are detected in acellular biological samples from animals with diseases. The presence of particular RNA molecules often correlate with the presence of the disease, or a risk for obtaining the disease. Often, the RNA molecules that correlate with the presence of a disease comprise repetitive sequences transcribed from repetitive DNA present in the animal germline, or sequences transcribed from regions of the animal genome that have undergone recombination or chromosomal rearrangements. Accordingly, the detection of these previously undetected animal nucleic acids is useful for risk assessment, early diagnosis, and continuous monitoring of diseases, particularly chronic illnesses. In addition, targeted destruction of cells from which these nucleic acids are being lost can be used to treat these diseases. The detection methods of the invention can also be used to monitor the success of treatment of disease.

The claimed methods of detecting sentinel nucleic acid sequences, e.g., DNA or RNA transcribed from repetitive DNA or chromosomal rearrangements, are not limited to acellular samples, but can also be performed using cellular samples. For example, tissue or cell samples, e.g., from brain, muscle, heart, liver, lung, stomach, colon, pancreas, immune system cells, or skin, can be analyzed for the presence of particular nucleic acid sequences associated with a disease such as a spongiform encephalopathy, e.g., bovine spongiform encephalopathy (BSE). In some embodiments the sentinel nucleic acid sequence comprises a repetitive DNA sequence, or an RNA transcribed from a repetitive DNA sequence, that has at least 70%, often at least 80%, 85%, 90%, or 95% sequence identity to a human repetitive DNA sequence, such as an Alu repeat.

The methods of the invention are particularly useful for risk assessment and diagnosis of neurological diseases, particularly, neurodegenerative diseases. These diseases include spongiform encephalopathies, also commonly referred to as transmissible spongiform encephalopathies (TSEs), such as scrapie in sheep, BSE, and encephalopathies of deer, elk, mink, and the like. The methods can also be used for the diagnosis of other diseases including transmissible diseases such as papillomatous digital dermatitis, foot-and-mouth disease, porcine reproductive and respiratory syndrome, and swine vesicular disease. Chronic diseases in animals can also be detected using the methods of the invention. Such diseases include not only neurodegenerative diseases, but also cancer, autoimmune diseases, and metabolic diseases.

In the current invention, the presence of one or more disease-associated target nucleic acid sequences, e.g., target RNA molecules in an acellular sample from an animal suspected of having a disease, is an indicator of the presence of the disease or of an increased risk for getting the disease compared to control animals. For example, the methods of the invention can be used for assessing whether an animal such as a cow has BSE.

BSE is clinically characterized by increasing perturbation of central nervous function in the affected animal, ultimately leading to severe symptoms, e.g., an inability to stand, forcing the sacrifice of the animal. In contrast to other mammalian TSEs, the bovine form does not appear to be associated with a mutation in the prion gene, but is caused by a post-translational misfolding of the prion protein, which leads to aggregation in the central nervous system. Attempts to develop antibodies specific for misfolded prions have failed thus far; the diagnosis is based on the fact that misfolded prions have enhanced resistance to protease K digestion. As disease-specific prion accumulation in the plasma or blood of animals has not been identified, the diagnostic target has been the brain stem. Therefore, there is an urgent need to define a blood-borne marker for TSEs so that the disease can be diagnosed in living animals.

In some embodiments of the invention the target sequences are sequences found in chromosomal repetitive elements that are homologous to those found in the human genome. Without wishing to be bound by theory, it is believed that nucleic acids that originate from particular chromosomal regions (e.g., repetitive sequences) are preferentially released from diseased or damaged cells early in or during the disease process. The nucleic acids can be released as a result of a number of events including contact with physical agents, including trauma, heat, cold, radiation and electrical shock; exposure to chemical agents and drugs, including aflatoxins, organophosphate and organochloride poisons (e.g., pesticides and nerve gas agents, nitrogen mustards), other chemical warfare agents, benzene, cigarette carcinogens, digoxins, dioxin, biotoxins and many other synthetic chemicals and drugs; exposure to UV light, radioactive particles, and other cell damaging radiation; exposure to infectious agents, including rickettsiae, bacteria, viruses, fungi, and parasites; immunological reactions that can cause cell injury; genetic alterations, such as chromosomal alterations or specific mutations in genes; oxygen deprivation, which occurs as a result of ischemia, inadequate oxygenation or loss of other oxygen carrying capacity of the blood; and nutritional imbalances. (See, e.g., Cotran R S, Kumar V & Robbins S L. in Robbins Pathologic Basis of Disease. 6th edit., Saunders, 1998).

### Repetitive DNA

Repetitive DNA includes tandemly repeated DNA elements and interspersed repetitive elements (summarized, e.g., in Purandare and Patel, supra). The tandem repeats are categorized based on the size of the blocks or arrays of tandem repeats. The categories are: satellite DNA (0.1 to over 2 Mb), minisatellite DNA (0.1-2.0 kb), and microsatellite DNA (about 150 bp). These categories can be further sub-classified based on the size of the repeat unit. Minisatellite DNA includes telomeric DNA, variable number tandem repeats (VNTRs), which have been implicated as hotspots for homologous recombination in mammalian cells, and hypervariable minisatellite DNA. Microsatellite DNA consists of small arrays of tandem repeats usually 1-4 by units that are interspersed throughout the genome, usually in blocks of less than 150 base pairs.

Repetitive sequences can also be categorized into long interspersed elements (LINEs) and short interspersed elements (SINEs) (see, Wilkinson et al. in The Retroviridae Vol. 3, J. A. Levy (ed.), pp 465-535, Plenum Press, New York (1994)). In humans, Kpn elements are examples of LINEs, whereas Alu elements are examples of SINEs. In human, the Alu repeat containing a 280 by repeat unit occurs approximately once every 4 kb in the human genome. Mispairing between such repeats has been shown to be a frequent cause of deletions and duplications. Breakpoints of disease causing deletions have also been clustered within Alu sequences. Similar short repetitive sequences occur in the genome of other animals and also can cause chromosomal rearrangements, e.g., deletions, duplications, breakage, translocations, and the like.

LINES, unlike SINEs, contain open reading frames encoding proteins with reverse transcriptase activity. Both LINES and SINES are examples of retroposons, which are a subcategory of retroelement, that is, a transposable element in the genome that transposes via an RNA intermediate. Retroposons are distinguished from retrotransposons (also referred to as endogenous retroviruses) by the absence of long terminal repeats (LTRs). The relationship between human endogenous retroviruses and various disease states as well as diagnostic detection of antibodies to human endogenous retrovirus antigens is discussed in WO 95/32311

As noted above, fragile sites may contain repeated sequences. Thus, in some embodiments of the invention, repetitive sequences or fragments thereof are detected in the invention. Repeated sequences are known to contain sequences that bind nuclear proteins and are effective in regulating gene expression. Evidence indicates that mobile elements such as segments of repetitive DNA (e.g., LTRs from retroviruses and repetitive sequences) have inserted in various sites in the genome and have affected regulation of gene expression (see, e.g., Britten et al. Proc. Nat. Acad. Sci. USA 93:9374-9377 (1996) and Deininger et al. Molecular Genetics and Metabolism 67, 183-193 (1999)). Without wishing to be bound by theory it is believed that alteration of these sequences by insertion of retroelements or genotoxic agents may lead to altered expression of sequences within the genome.

### Detection of Nucleic Acids

The nucleic acids detected in the methods of the invention are typically from about 40 nucleotides in length to several thousand nucleotides in length. Usually, the nucleic acids are from about 200 to about 1500 nucleotides.

The present invention is also directed to the detection of novel viral nucleic acids and recombinants between these viral nucleic acids and other nucleic acids from diverse origins. In some embodiments the viruses are members of the picornaviridae, such as enteroviruses. The complete nucleotide sequences of various enteroviruses are available in the scientific literature and in databases such as GenBank. Using this information, one of skill can design appropriate primers and probes targeting desired regions of a picornavirus genome. Selection of the primers used in the invention is based on what target sequences are being detected. In the case where contaminating viruses are being detected (e.g., in a vaccine preparation) primers which specifically hybridize to any region of the virus genome can be used.

If chimeric virus and animal sequences are being detected, a primer specific for animal sequences is used in combination with a primer which hybridizes to sequences conserved in a picornaviral genome.

The diagnostic methods of the invention typically rely on a method of amplifying the target nucleic acid from an acellular biological fluid (e.g., serum or plasma). PCR amplification of the target nucleic acid is typically used. One of skill will recognize, however, that amplification of target sequences in a sample may be accomplished by any known method, such as ligase chain reaction (LCR), Qβ-replicase amplification, transcription amplification, and self-sustained sequence replication, each of which provides sufficient amplification.

The PCR process is well known in the art and is thus not described in detail herein. For a review of PCR methods and protocols, see, e.g., Innis, et al. eds. PCR Protocols. A Guide to Methods and Application (Academic Press, Inc., San Diego, Calif. 1990). PCR reagents and protocols are also available from commercial vendors, such as Roche Molecular Systems.

The nucleic acids detected can be DNA or RNA molecules. In particular embodiments of the invention, RNA molecules are detected. The detected RNA molecules can also be RNA transcribed from genomic sequences, but which do not encode functional polypeptides. The first step in the amplification is the synthesis of a DNA copy (cDNA) of the region to be amplified. Reverse transcription can be carried out as a separate step, or in a homogeneous reverse transcription-polymerase chain reaction (RT-PCR), a modification of the polymerase chain reaction for amplifying RNA. Methods suitable for PCR amplification of ribonucleic acids are described in Romero and Rotbart in Diagnostic Molecular Biology: Principles and Applications pp. 401-406, Persing et al. eds., (Mayo Foundation, Rochester, Minn. 1993); Rotbart et al. U.S. Pat. No. 5,075,212 and Egger et al., J. Clin. Microbiol. 33:1442-1447 (1995)).

The primers used in the methods of the invention are preferably at least about 15 nucleotides to about 50 nucleotides in length, more preferably from about 15 nucleotides to about 30 nucleotides in length.

To amplify a target nucleic acid sequence in a sample by PCR, the sequence must be accessible to the components of the amplification system. In general, this accessibility is ensured by isolating the nucleic acids from the sample. A variety of techniques for extracting nucleic acids, in particular ribonucleic acids, from biological samples are known in the art. As noted above, the samples of the invention are acellular biological fluids.

The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid (amplicon).

In the preferred embodiment of the PCR process, strand separation is achieved by heating the reaction to a sufficiently high temperature (˜95° C.) for a sufficient time to cause the denaturation of the duplex but not to cause an irreversible denaturation of the polymerase (see U.S. Pat. No. 4,965,188). Template-dependent extension of primers in PCR is catalyzed by a polymerizing agent in the presence of adequate amounts of four deoxyribonucleoside triphosphates (typically dATP, dGTP, dCTP, and dTTP) in a reaction medium comprised of the appropriate salts, metal cations, and pH buffering system. Suitable polymerizing agents are enzymes known to catalyze template-dependent DNA synthesis. In the present invention, the initial template for primer extension is typically first strand cDNA that has been transcribed from RNA. Reverse transcriptases (RTs) suitable for synthesizing a cDNA from the RNA template are well known.

PCR is most usually carried out as an automated process with a thermostable enzyme. In this process, the temperature of the reaction mixture is cycled through a denaturing region, a primer annealing region, and an extension reaction region automatically.

Nucleic acids that are associated with a disease can also be detected by detecting the presence in a sample, e.g., acellular fluid, of a biological activity associated with the nucleic acid. The biological activity is often a catalytic activity such as a catalytic activity associated with an RNA or single-stranded DNA molecule, e.g., nuclease activity, nucleotidyl transferase activity, or esterase activity (see, e.g., Kruger, et al., Cell 31:147-157, 1982; Crimi et al., Proc. Natl. Acad. Sci. USA 95:2233-2237, 1998; U.S. Pat. Nos. 6,326,174; 4,987,071; 6,221,661). The single-stranded nucleic acid can thus act as an artificial enzyme, catalyzing such chemical reactions as phosphoester transfer (e.g., U.S. Pat. No. 6,326,174) phosphoester formation (e.g., Cuenoud et al., Nature 375:611-614, 1995) prophyrin metalation (e.g., Li & Sen, Nat. Struct. Biol. 3:743-747, 1996), phosphoramidate cleavage (e.g., Burmeister et al., Angew. Chem. Int. Ed. Engl. 36:1321-1324, 1997), and nucleic acid cleavage (e.g., Carmi et al, Chem. Biol. 3:1039-1046, 1996; Kruger et al., supra, and U.S. Pat. Nos. 4,987,071 and 6,221,661). Nucleic acid cleavage assays and other assays of single-stranded nucleic acid catalytic activity are known in the art (see, e.g., the references cited herein).

Other biological activities of the target nucleic acid can include the ability to regulate gene expression when introduced into a cell or the ability to alter cell surface characteristics. For example, RNA molecule such as the HIV genome induce a wide variety of biological effects (see, e.g., Chapters 60 and 61 in Fields VIROLOGY 3rd Edition, Fields et al, Eds; Lippincott, Williams & Wilkins, 1996). The target RNA molecules described herein (e.g., sequences that are from germline sequences in the host animal, such as sequences that are rearranged in comparison to normal, or chimeric nucleic acid sequences comprising both germline sequences and sequences from another organism) can also exhibit similar biological effects.

The target animal nucleic acids of the invention can also be detected using other standard techniques, well known to those of skill in the art. Although the detection step is typically preceded by an amplification step, amplification is not required in the methods of the invention. For instance, the nucleic acids can be identified by size fractionation (e.g., gel electrophoresis). The presence of different or additional bands in the sample as compared to the control is an indication of the presence of target nucleic acids of the invention. Alternatively, the target nucleic acids can be identified by sequencing according to well known techniques. Alternatively, oligonucleotide probes specific to the target nucleic acids can be used to detect the presence of specific fragments.

As explained in detail below, the size of the amplified fragments produced by the methods of the invention is typically sufficient to identify the presence of one or more bands associated with a particular disease. Thus, in some embodiments of the invention, size fractionation (e.g., gel electrophoresis) of the amplified fragments produced in a given sample can be used to distinguish the fragments associated with a particular disease. This is typically carried out by amplifying a control with the same primers used to amplify the sample of interest. After running the amplified sequences out in an agarose or polyacrylamide gel and staining, the nucleic acid, e.g., with ethidium bromide or other stains such as fluorescence dyes, e.g., SYBR Green™ (Molecular Probes) according to well known techniques (see, Sambrook et al.), the pattern of bands in the sample and control are compared. The presence of different or additional bands in the sample as compared to the control, is an indication of the presence of a band associated with a disease.

Sequence-specific probe hybridization is a well known method of detecting desired nucleic acids in a sample comprising cells, biological fluid and the like. Under sufficiently stringent hybridization conditions, the probes hybridize specifically only to substantially complementary sequences. The stringency of the hybridization conditions can be relaxed to tolerate varying amounts of sequence mismatch. If the target is first amplified, detection of the amplified product utilizes this sequence-specific hybridization to insure detection of only the correct amplified target, thereby decreasing the chance of a false positive caused by the presence of homologous sequences from related organisms or other contaminating sequences.

A number of hybridization formats are well known in the art, including but not limited to, solution phase, solid phase, oligonucleotide array formats, mixed phase, or in situ hybridization assays. In solution (or liquid) phase hybridizations, both the target nucleic acid and the probe or primers are free to interact in the reaction mixture. Techniques such as real-time PCR systems have also been developed that permit analysis, e.g., quantification, of amplified products during a PCR reaction. In this type of reaction, hybridization with a specific oligonucleotide probe occurs during the amplification program to identify the presence of a target nucleic acid. Hybridization of oligonucleotide probes ensure the highest specificity due to thermodynamically controlled two state transition. Examples for this assay formats are fluorescence resonance energy transfer hybridization probes, molecular beacons, molecular scorpions, and exonuclease hybridization probes (reviewed in Bustin S M. J. Mol. Endocrin. 25:169-93 (2000)).

In solid phase hybridization assays, either the target or probes are linked to a solid support where they are available for hybridization with complementary nucleic acids in solution. Exemplary solid phase formats include Southern hybridizations, dot blots, arrays, chips, and the like. In situ techniques are particularly useful for detecting target nucleic acids in chromosomal material (e.g., in metaphase or interphase cells). The following articles provide an overview of the various hybridization assay formats: Singer et al., Biotechniques 4:230 (1986); Haase et al., METHODS IN VIROLOGY, Vol. VII, pp. 189-226 (1984); Wilkinson, IN SITU HYBRIDIZATION, D. G. Wilkinson ed., IRL Press, Oxford University Press, Oxford; and NUCLEIC ACID HYBRIDIZATION: A PRACTICAL APPROACH, Hames, B. D. and Higgins, S. J., eds., IRL Press (1987).

In preferred embodiments, the solid phase hybridization assay is in an array format, which provides the ability to identify the presence of multiple target animal RNA molecules in a sample. In one embodiment, high density oligonucleotide analysis technology (e.g., GeneChip™) is used to identify the presence of sentinel animal RNA sequences, e.g, sentinel sequences present in an acellular fluid. The sequences on the array can be up-regulated in diseased animals in comparison to normal controls, down-regulated in the diseased state, or a combination of both up-regulated and down-regulated sequences. For example, in the case where the RNAs being identified are linked to a known disease, they can be used with GeneChip™ as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

The hybridization complexes are detected according to well known techniques and are not a critical aspect of the present invention. Nucleic acid probes capable of specifically hybridizing to a target can be labeled by any one of several methods typically used to detect the presence of hybridized nucleic acids. One common method of detection is the use of autoradiography using probes labeled with ³H, 1²⁵I, ³⁵S, ¹⁴C, or ³²P, or the like. The choice of radioactive isotope depends on research preferences due to ease of synthesis, stability, and half lives of the selected isotopes. Other labels include compounds (e.g., biotin and digoxigenin), which bind to antiligands or antibodies labeled with fluorophores, chemiluminescent agents, and enzymes. Alternatively, probes can be conjugated directly with labels such as fluorophores, chemiluminescent agents or enzymes. The choice of label depends on sensitivity required, ease of conjugation with the probe, stability requirements, and available instrumentation.

The probes and primers of the invention can be synthesized and labeled using well known techniques. Oligonucleotides for use as probes and primers may be chemically synthesized according to the solid phase phosphoramidite triester method first described by Beaucage, S. L. and Caruthers, M. H., 1981, Tetrahedron Letts., 22(20):1859-1862 using an automated synthesizer, as described in Needham-VanDevanter, D. R., et al. 1984, Nucleic Acids Res., 12:6159-6168. Purification of oligonucleotides can be performed, e.g., by either native acrylamide gel electrophoresis or by anion-exchange HPLC as described in Pearson, J. D. and Regnier, F. E., 1983, J. Chrom., 255:137-149.

The present invention also provides kits, multicontainer units comprising components useful for practicing the present method. A useful kit can contain probes for detecting the desired target animal nucleic acid. In some cases, the probes can be fixed to an appropriate support membrane. The kit will also contain primers for RT-PCR. Other optional components of the kit include, for example, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. In addition to the above components, the kit can also contain instructions for carrying out the present method.

The invention provides methods of treating illnesses in animals, including chronic illnesses such as bovine wasting disease or cancer. Generally, the therapeutic methods rely on therapies designed to significantly reduce the presence of acellular nucleic acid complexes or limit their expression, or to selectively destroy cells from which nucleic acid complexes are being released. In many cases, such cells are dysplastic, particularly in the case of cancers. In some cases, such cells may be part of the immune system, e.g. lymphocytes, macrophages, follicular dendritic cells, eosinophils, bone marrow cells and the like. Thus, compounds that can selectively destroy such cells or limit the release of selective RNA complexes can be used to inhibit the disease process. For instance, compounds that selectively induce apoptosis in target dysplastic or neoplastic cells can be used in this approach. Examples of such compounds are sulindac-derived compounds such as sulindac sulfone, a non-steroidal anti-inflammatory drug. The growth inhibitory effect of sulindac sulfone results from the ability of that compound to selectively augment cell death through apoptosis, rather than by arresting the cell cycle.

Any number of anti-neoplastic compounds and therapies known to those skilled in the art can be used in the present invention. Such compounds work by a number of mechanisms including inhibition of purine or pyrimidine synthesis, inhibition of deoxyribonucleotide synthesis, cross-linkage of DNA, inhibition of microtubule formation and the like. For a description of a variety of chemotherapeutic agents, see, Principles of Internal Medicine 12th ed. pp 1587-1599 Wilson et al. (eds.), McGraw-Hill, Inc. 1991)

Suitable pharmaceutical formulations for use in the present invention are found in Remington's Pharmaceutical Sciences, Mack Publishing Company, Philadelphia, Pa., 17th ed. (1985). A variety of pharmaceutical compositions comprising compounds and pharmaceutically acceptable carriers can be prepared.

Injectable preparations, for example, sterile injectable aqueous suspensions may be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

The pharmaceutical compositions containing the compounds can be administered for therapeutic treatments. In therapeutic applications, compositions are administered to a patient already suffering from a disease, as described above, in an amount sufficient to decrease and preferably cure or at least partially arrest the symptoms of the disease and its complications. An amount adequate to accomplish this is defined as “therapeutically effective dose.” Amounts effective for this use will depend on the compound being administered, the severity of the disease, the weight and general state of the patient, and the judgment of the prescribing physician.

### EXAMPLES

### Example 1

### Identification of Extracellular, Nonviral RNA in Animal Serum

The following example demonstrates the presence of extracellular, nonviral RNA in normal fetal bovine serum, horse serum, and goat serum. PCR primers that can be used include 18S primers, for example, from Ambion. RT-PCR is performed as follows: RNA from the serum sample was extracted using TRIZOL LS reagent (Gibco BRL, Gaithersburg, Md.), and precipitated with 10 μg of RNase-free glycogen as a carrier. Both methods are typically performed according to the protocols of the manufacturer.

The precipitated RNA is washed once with 70% ethanol by centrifugation at 4° C., resuspended in 10 μl of Rnase-free distilled water, and added to 17 μl of the RT mixture (GeneAmp RNA PCR kit; Perkin-Elmer, Norwalk, Conn.) containing MgCl₂(5 mM), 1×PCR Buffer II, Rnase Inhibitor (2.5 U), MuLV Reverse Transcriptase (2.5 U), random hexamer primers (2.5 μM), and 1 mM each of dATP, dGTP, dCTP and dTTP. The mixture is incubated for 10 minutes at 22° C., 30 minutes at 42° C., 5 minutes at 95° C. using a Perkin-Elmer Thermocycler. The RT mixture is then added to the top PCR mixture of a Hot Start PCR reaction using a melted Ampliwax bead (Perkin-Elmer, Norwalk, Conn.) as the barrier. The 70 μl top PCR mixture contains 1×PCR Buffer II and Amplitaq (2.5 U). The 30 μl bottom PCR mixture contains 1×PCR Buffer II, 2 mM MgCl₂, and the appropriate primer pairs (15 μM). After 35 cycles (1 min at 94° C., 2 min at 48° C., and 1 min at 72° C.), 8 μl of the PCR mixture is subjected to electrophoresis using a Pre-Cast 4-20% gradient or a 6% polyacrylamide gel in TBE Buffer (45 mM boric acid, 1 mM EDTA) (NOVEX, San Diego, Calif.) for 45 minutes and 60 minutes, respectively, at 200 volts. After electrophoresis, the gel is stained in a 0.5 μg/ml solution of ethidium bromide solution for 20 minutes and the bands photographed under UV light.

Such an analysis performed using 18S ribosomal RNA primers from Ambion as PCR primers for an RT-PCR reaction showed that a single band is present in fetal bovine, goat, and horse serum samples. The band was subcloned and analyzed by DNA sequencing, which identified the band as corresponding to a region of the 18S ribosome that is conserved across species.

Thus, RNA molecules were identified in acellular biological samples from animals.

### Example 2

### Repetitive Sequences in RNA in Serum of BSE Animals

This example shows that RNA molecules detected in a BSE animal contain sequences that include repetitive DNA.

Bovine blood was collected from a cow (designated as “200”) that had a confirmed diagnosis of BSE and a healthy herd mate (designated as “180”) via venipuncture and stored at 4° C. for not more than 24 hours. Serum was collected using centrifugation at 2000 g for 10 minutes at 8° C., immediately aliquoted and stored frozen at −20° C.

RNA was isolated from 250 μL of unfractionated serum using solid phase adsorption followed by RNAse protected DNAse digestion (Nucleospin RNA extraction kit, ClonTech). The total RNA was then used for full length cDNA construction using a SMART kit (ClonTech), referred to as SMART amplified cDNA.

SMART amplified cDNA (2 μL) was used for cloning into the vector TOPO-TA PCR II (Invitrogen) and transformed into TOP 10 electrocompetent cells according to the manufacturer's instructions. Cells were plated on x-GAL LB-agar plates with Kanamycin. Positive clones were selected for sequencing.

Each recovered clone was sequenced using an IRD-700 labeled M13 forward and a IRD-700 labeled M13 reverse primer using a model 4200 LICOR DNA sequencer. The final sequence was checked for the presence of the SMART-specific sequence to ensure insert integrity.

The sequences of 11 clones from the SMART-amplified cDNA of the healthy cow (“180”) and of 12 clones from the BSE cow (“200”) were obtained and used to search the nucleotide databases using the BLAST engine at http://www.ncbi.nlm.nih.gov/BLAST/. Search conditions were set with: word size=15, Expect-10000 and Low Complexity Filter=off.

The following clones showed sequence homology to entries in the databases. The sequences of two of the clones from the BSE cow contained regions corresponding to human Alu sequences; one of the clones contained sequences homologous to the sequence contained between two Alu sequences.

| | |
|---|---|
| Fragment Size | Comments |

Cow “180” (Healthy):

| | | |
|---|---|---|
| 180/7RE | 361mer | not in a repeat region |
| | | sequence unknown |
| 180/9RE | No Significant Homologies | |
| | Found | |
| 180/12RE | 50MER | Cloning Vector |
| 180/18RE | 66mer | Poly A |
| 180/19RE | 44mer | Cloning Vector |
| 180/21RE | 36mer | CT repeat |
| 180/22RE | No Significant Homologies | |
| | Found | |
| 180/28RE | 33mer | rRNA |
| 180/29RE | 50mer | Cloning Vector |
| 180/30RE | No Significant Homologies | |
| | Found | |
| 180/31RE | 49mer | Cloning Vector |

Cow “200” = BSE:

| | | |
|---|---|---|
| 200/4 | No Significant Homologies | |
| | Found | |
| 200/5 | 69mer | Cloning Vector |
| 200/6 | 72mer Alu | Two Alu sequences |
| 200/7 | No Significant Homologies | |
| | Found | |
| 200/8 | No Significant Homologies | |
| | Found | |
| 200/9 | 711mer | Sequence found |
| | | between 2 Alus |
| 200/10 | No Significant Homologies | |
| | Found | |
| 200/11 | 411mer | Genbank Submission- |
| | | no background |
| | | information |
| 200/12 | 280mer Alu | Alu region |
| 200/13 | 39mer | Cloning Vector |
| 200/14 | 49mer | Cloning Vector |
| 200/15 | 41mer | L1PB2 region |

The sequences obtained from the clones are shown below. The italicized, bolded regions correspond to regions of homology in the database.

Normal Cow “180” Clone #:
180T7REV

(SEQ ID NO: 10)

GAGCTCGGATCCACTAGATAACRGCCRCCAGTGTGCTGGAATTCGC
CCTTMAGCAGTGGTAACAACGCAGAGTAC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

CCCGCGTACTCTGCGTTGTTCACCMCTG
CTTTAAGGGGCAAAWTTCTACNNATNYCCCCCCCCCGGGGGNCCGC
NCNGNNCNTNMATCGGGGGGCCMAATTSCCCCCTNNNGNRAGBTCN
TANAACAANNCCGCGGSGSCGG
180T9REV

(SEQ ID NO: 11)

TCTCNGATCACTANTTCGGCCGYCANTTTGCTNGAWTTCGCCCTTM
AGCAGTGGTAACAACGYAGAGTAYTTTTTTTTTTTTTTTTTTTTNT
TTTWHCAATYATTGGATYTHCGNRAGWTGTTTCGTCAGAATTATCA
CCATTGTYATATTCAAAACYAATAAKKTTAAYWAAGTAATTGTYAC
TTACAGCGNCAATTKATTCTTTTGCAWCTAAATTGTTTTCAATKAT
TACWTCTTTTATTTTHTTCATTWTTTCGTWCCCTTTATTCMTACCT
TTDATTTYTTTWWGGTCNTCCATCTTTTANGCCCYTNNTCKGTNTA
AAGGTTCTAGNGGATCTTCGTAAATTTTTTGTTTTTTTTTT
180T12REV

(SEQ ID NO: 12)

[symbol]

[symbol]

[symbol]

[symbol]

[symbol]

[symbol]

[symbol]

AGCAGTGGTAACAACGCAGAGTACTTTTTTTTT
TTTTTTTTTTTTTTTTTTCAATCATTGGTTCTTCGTGARATTTTTC
GTCAAAATTATCACCATTGTMATATTCAAAACCAATAAGGTTAACA
AAGTAATTKTCACTTACAGCGTCAATAGATACTTTTGCAACTAAAT
GGTTTACAATGATAACATCTGTAATTTTATTCATGATATCGTACCA
TTTATTCATACCTTTGATTTCTTTAAGGTCATCCATCGTTAAAGCA
CCTAAACGATATAAAGGTTCAGTGAACTTCGCAAATTCCATGAAAT
TTGTATCTTCTGGATTGAATGTTGATACAAATKTTTCGTTCAGTGG
AATTTTTCCNNCTTNYAANGGNCNNGANNAAAAAGNTTNGCCTTCA
ATACCATCAATTNCAAAAAAAAGNCANACNGACTGTATAAAGCMCG
GACCACTGCGNNNTANAACNNGAAAANGTTTTTTYCTAAGTTTCAT
CMCAGTCAANATNNANCGAAAGGTGGTTTNGNCANNNTCGYVSGAA
NNGGGGGCCCNGNNANNNNACCCNAMNCNNNGNNNNGAAANAAAAA
MCCAMCCCGG
180T18REV

(SEQ ID NO: 13)

CGGATCCACTAGTAACGGCCGCCAGTGTGCTGGAATTCGCCCTTAA
GCAGTGGTAACAACGCAGAGTACGCGGG

[symbol]

[symbol]

[symbol]

[symbol]

[symbol]

[symbol]

[symbol]

[symbol]

[symbol]

AAARAATTRTGAGCCGCATTANTNNNNNTTTNTNNN
NTATWTNNTTNNNANNNNNNTNNTTTTTTTTNAAAATTNTTCYCCC
CBTTNNTCMCYTNTGGGGTKGGGGGTTTTCCTCCCTTTTCCCCTTT
TTTTNGGGGCTTTTNNTTGGGCSGCYKCCCAGGGGCSSCGCGGTTN
TTNTNNTTCCANNNNNGGGGGGTNTTGNNNATTTTTGTGCTBTTNN
NNNNTNNCC
180T19REV

(SEQ ID NO: 14)

[symbol]

[symbol]

[symbol]

[symbol]

[symbol]

[symbol]

MAGCAGTGGTAACAACGCAGAGTWCTTTTTTTTTTTTTTTTTTTTT
TTTTTTTTATYCACTTCTCCAAAAAGAAGAATGATTAAMTTTATTT
AGAATAATAAACAAATGAAATTGATCTTTACTTAGATCTCGGCCAA
TTCACGGAGACGAMGAACAGTGGAACGGACARTGGCAGCARCAGTA
GTGGAGACGGTGTAARCACCACCAGCCATGGTCTTCTTACAACCCT
TACACTTCCAGATACCAACAGCGGTACSTTGNACAGCATCCTTWCC
ACAGAAGGTACAAGTGTAGGTGGAATKTTGAGTGATTTCCATCATC
TTGACAGTCTTTCTGGGAGAGGCACCATAACGGAGTACCGTATTTA
CCGGTGATTCCSCCCTTCTTAKTKCGCTTTGCCATTTTAGATTAAA
TACCAAAGAGGAGCCCCGCGTACTCTGCGTTGTTACCACTGCTTAA
GGGGCGAAATTCTACAGATTTMCTTCACNCTGGGGGCGCTCGNAGC
NTACATATAGNGGGCACCATTTCCCCCTATNNANNNTCANTATNAC
NNNNCNNNNGGGCGGGCGGKGGGKCCAGCGTCATNNCTAGGANNCC
CCTGGGCRTTNCCCNNCWTTNTTCGCCTTTGGAGANNNNTCCCCCT
TTTTAGCCNNNTTCGGCGTTAATTARCCGAGNGGAGGCCMSCCCCC
GCTCGCCCCTCAMCACACNGTTACGCGCCATNNTTGGAGCSGNATG
CCGCGCGCCCWTTNGGCGCGC
180T21REV

(SEQ ID NO: 15)

GTTCNGGCCGNCTNCCGTTGGTGGCTNTATGNTTCCATSTGSGTCC
CTGCTNTTNCCGCCAGTGCTCTCCGTGTTTGGCTNTCCCNCNCGCT
CTGBACAAAASAAAGCCAAACATCAAAAGGCCGGCNNTGCNTAAAG
CCAACNATANAGCCCTCTCTCNTCCGTCNNCGCCGTNCCNNGCNNT
GCGGCNNGCCKCNGCGGNGNNNNCTCTGCGGNNTCNTGCGGGNNGN
CTNTTTTTTCTATGTGCCCNCGCCTCTNGCCAGCAGTNTNTNTTNC
TCNNTTCTGGCCTCTTTCTTGCNNTTTTCTTTGTGGTCNCTTTNCC
KGCCNTTNNCCCTTNTCKTNCNCNCT [symbol] [symbol] [symbol] [symbol] [symbol]
CKCTTCGCNTCNTNNTNTNTNNTNTNN
NCGNCGCCTTGCTTTGNTTTNTTTTGTCGGNTTNCTGTGTNTTTNT
GTTNTGGGGNTTTGNTNTCCTGCNNTCSCNTTTCCNCTTTTGTGGG
NCTTTTGGCNGTTTTGTTNNYNCNGTGTGTNTCCNTGGGCNTTGNN
NCGTTNNNNTGTTSNYCTNTCTCNSKTTNYNNYNNYNYNNNCTTTN
TCTNTTNNNCTTTGCTNTCGCTGTSNGNCTGCTSTNGTTSNTGNCG
CTCTNTTTCTTTGTNNTTNSNTTTTCTCTNCTCTCTCTTCTCNCTC
GTCGNGNSNSNNNCTCTCTCTCTNYGTGTGNNYCCTGTGTNCCKCT
CTGTCTGNTNTTTCGTCKCNCNNTTTTTNCCGCTNTGTNNTNTTTN
NCCYCTCTCNTCTNNTNTTGTNN
180T22REV

(SEQ ID NO: 16)

GTWAAGGCGAMTACCGATGGWGGMTNTATGATTCCATGAGGGCTAC
CTGACAAATNCCGCMGATGACTCAAAGAANATAAAANNNAANANNN
NAAGKABATTTTTCGARCGGCCTRTCCNTTGGCCTTGACNATATAA
TNGGACAGAAAATAAYMMMYCTAGAAAMATMCMCACNAGATAGCCT
WTGANTTTTCGCCASMMYCCCCCYACGCGAARACGCANNGCGGGCC
CGACCGGCGAGANGGCATCCGGAKRGGMCCCACCATGCANGGGGGT
ATGCCCKGAGGGGNTNACACGCTGGGACANTTTGGCCGGAAGGNCC
TGGGGGGAATGAAGAAAACAACNGCAAAGMATGGGCCAGGAAAGRG
ANGGANNGGMGANTCCCAARCNAAAGATWWCCGCATARNTGANTTT
AANNNTTCCMGAAAAACACCCCCNANAAMCCTCCAACCAACCGATC
NNAAGGCAAGCANGAAGGGRCGATNGCCGGGGGGTRTACGAGGTGT
NNNNCCGCGATGAACCTATTTAYCCGGAKKTAACSGGNCGSGCAAT
TTTTGGKAAGNTCTCCAAAAMAAACAATTTGGTCCAAG
180T28REV

(SEQ ID NO: 17)

GAGCTCGGATCCACTAGTAACGGCCGCCAGTGTGCTGGAATTCGCC
CTTAAGCAGTGGTAACAACGCAGAGTACTTTTKTTWTTTTTTTTTT
TAANTTTCAAAGTAAAGGTACCTGGTTCCCTGCTGCCGGAACAACA
GATCACCAAGAGGAAAGACCCAGCAAGACAGWACTCGCCGTGAGGC
ANCCGCCTGTCCAGGTCMAAGG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

TACGACGNAWAAGGSCGNATATTG
TTACAAATTGTCACTACCTCCCCGNGNNGGCGANNGGGGAANNNGC
GCGCCNNNNGNMTTCGTNGGANGNGGNAGCCANNTCNCAGGNNCCN
NNCCGGAATNGAANCGNTANNCCCGNNAANNCGNNNNAANNANGGN
NGG
180T29REV

(SEQ ID NO: 18)

[symbol]

[symbol]

[symbol]

[symbol]

[symbol]

[symbol]

[symbol]

AGCAGTGGTAACAACGCAGAgTAGCGGGACTGGAAGTTGAGAG
TACAATGTCTACAGGAAGGAATTCATGCATGGTATGCTGCAGTGTG
TTGAGTGCGTTGTTGTAGGGGTTACCGCGTGTGTGGCAATTGTgAT
GATGTTGGCTTGTAGCAAATgACCAGGAGTTGATGGGTGTTGGTGA
TTGTWAGACAAGCATGGTGCTGGTCGATTAGTATTTGTAATAGCTG
TGTCTAAAAAAAAAAA
180T30REV

(SEQ ID NO: 19)

CGGATCCACTAGTAACGGCCGCCAGTGTGCTGGNAATTSGCCCTTD
AGCAGTGGTAACARCGCAGAGTACGCGGGTKATRRRAGACCAAATR
RDATATAADRDGCAGTAAAADRDRRRRDDDKRATKRDNRGRKACAT
AAAGATAAADKGRGGTGAAATRRAAGGGGGNTNGGGGBKKTTKKGG
NKNNTTCNGAGGTTNNGRCARAAGTTRATGGGCAGAAAAGGKGTGN
TNTGNCNKTNNTGBKTKGGGCCNNNCSCBTCNNNNNCCTNNNNCTT
GNGNGBKGGNKNNSCGGNNKSCNGNTTCCCNNNNKBKKKKTKKBGC
KNANTCNNTNNNACNNCCGNGBGGGNGNGNGGKGNGGKCNNCCCCC
NCCNNNNCCNNNCCCGGGSGGNGGTGNTNNGATTGNTNGSGG
180T31REV

(SEQ ID NO: 20)

[symbol]

[symbol]

[symbol]

[symbol]

[symbol]

[symbol]

[symbol]

AGCAGTGGTAACAACGCAGAGTACGCGGGCCAGTGATATAT
TCAGATCCTATTCCTTCAATGAGATTTCCAACCATTCAGTTTCAAT
ATGTTCAAAAAAGAAGATTCAAGTAATCATGCACATCAAACACTTT
TTTCACTGATCACCTCTTTTTTTTTATTCGCAAATCAAATCATCAC
TTTTTGTTATTTTCCAAAGGAGCATTTTTTTTCTCCTATATTTTAC
TAGGTTAATTTTAGTATATATTATTTATKATTTATKATKATKATCA
TCTGAKTTGGTGGTTCTCATGGCTGGTCTGGTTGGTGCTTACCTGG
TGATCTCTTGGGTTCTGTTCCTCTGGTCTTTTGATCTATGTCAAGT
GTAATTGGGCNCCTTTTTTGCTTACTGGTCTTTTGATACTCGCGAG
TTAGCCCTAGGCTTTTCTTTTTTTCGGCAGAATGTATACATTTGAT
GATGAGATTTATTTTNGAGGANTNATTCTATTCAGCTCACTTTCTA
CAACTTCAGCTTCAAACATTTTNAGNCGANGGGACANNGGNGNANN
NAAATTNNGNAANANCCANAANCNCACCANTNTGKRTKTNNGGAGR
ATCNCTNNAAGAGCNNTNCKBTNTNGNNNNNGAAGGGGAAGNCTTN
NANGGGGGGANTCNATGCNNNNNNGGGGNNNARAAAGTTNNGANNT
NCGTTNCGTTAAATGGNNNNNANNNGGNNGCNANTGGGGNNNNGNN
NGGNCAAACAAAACNNNC
BSE Cow “200” Clone #:
200T4REV

(SEQ ID NO: 21)

TCGNNTYTGANTCCNCTATATNYAGCYACCAGTKTNMTNGNNTTCA
GCMCTWTNGMTATRGTTNYTAAGANTTATTTCGKGNNTGKTTTTTN
CTCTNATTCTTNGAGAANNGTNTNNNTHTATCNCTNCTGGTTHTNA
NCNNAYTTGNCNCTGTTNNKGNTTNTCCCYNTHTTTNTTTTTTCAT
TNNGNCTCNTTTNCCTNNNNTGNANCNGGTNGNNNCCNTCNNNNTT
TNGGTNNCC
200T5UNI

(SEQ ID NO: 22)

[symbol]

[symbol]

[symbol]

[symbol]

[symbol]

[symbol]

[symbol]

[symbol]

[symbol]

[symbol]

CCSTTAAGCAGTGGTAACAACGC
AGAGTACGCGGGACAGTTCCKTTGGAATT ATAGCATAGAAATCGA
CTTCAAAATGGCTCAACGTGTTACTTACAGAAGAAGAAATCCATGT
ATGTTTATATCTAAGTATGACCAGGACTATTGAACTATAAGAATGA
AAGAGAAGAGCAAAGGACGGTGATACGGTCCAATGAGAAATTTATG
ACAAAATACAATACCAAATCTAACAAGATTAAGGTTGTTAAGACCS
CAGGTGGTAAATTAGTTGCCCAACACGTCAAGAAGCTCGCTTCTAG
ACCAAAGTGTGGTGACTGTGGTGATGSTTTACAAGGTATCTBTACT
TTAAGACCAAGAGAATACGCTCAAGTTTCTAAGACCCACAAGACCG
TCCAAAGAGCSTACGGTGGTTSTAGATGTGCTAACTGTGTCAAGGA
AAGAATTGTCAGAGCTTTSTCTGATCCGAGNACAAAAGATCGTTAA
GGGAGTGTTGAAAGAACAACAAGATAAGGCACAAAAGTCCGCCCAA
GGANGACCGGKAAGAAATANGTTAACTTAGGNTTGAMGCTTTGTTA
TATCTAGTTTTTGGTTTTRATGGTTCTTCTATGTAAATTTCTTTGT
CGTTTTAATACACATTTTTACGTTACAAAACGGCGCGCNCNNNNNN
NCTSGC
200T6REV

(SEQ ID NO: 23)

CRATCCCTAGATAAYRGCCAGCCANTTTGCTNGAATTCGCCCTTAA
KMWATAGTTACWAMGCAGAATACGCTGGGGGAKGYTTAATTTGCTG
ATAGAAAM [symbol] [symbol] [symbol] [symbol] [symbol] [symbol] [symbol] [symbol] [symbol] [symbol]
ATAGCGGTGAG
CTCACACCTWGTTATCCCANCNCTTTGGTAGGCAAAGGCTATTTGG
TTCWYATTTGTCANRATTTCTAKACCAAGCCTGGCCANCATAGTGA
AACMMAAATCATCWTAMATAAAATATTCTANNTTTTAACCACT

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

AMMTGKGGKNCAA
AACAATAMTCAGTCTCGNNNNNKNNNNTTTTT
200T7REV

(SEQ ID NO: 24)

GAATCCACTANATAACAGGCCGCCAGTGTGMTGGAATTCCCCCTTA
ANMAATAGTAACAAMGCAGAAWCGCGGGGATAAGGAAATACACAAA
GAAAWAWRAAAAMMAAAACAAAGGGATAAAAMTACCGTTTTAGATG
AACTTTTTCAGCAAACTTGGTATTACAGATAGGGAATGCCAAAGAG
AGGCTAGATTCTATTTATTCATCTTCATATAGCTGGGCTAAAGGGC
ATAGCAAAGGATTTTAATTTAGGTCATATGATTTAGGGGTTNGCAT
TTTTTACGGTACATAAATTTGGGGTCTGTRMGCATGWGTTNGCTAN
AGAGGATCTAAGCAGGCTATTCAGNNNTCATTNNNGNCTNNAGAAC
AAAAGAAGGSCAGGTGTAATGGCTCATGCCTATAATCCCAGCACTT
TAGAGAGGCCGAGGTRGAAGGATTTNTTTAGGGCAGGNTTTCAAGN
NCAGCCTGGNCNNACCGCNGNCCTTTTCTCTGCAAAAATTTTTATC
NATTAGCCANTTNTGGTGGTNCNTNCCTGTTCTTNGCTACCTTAAA
GNGGGSTGNNNNNNGGANAATTCNNCCTNNGGGTTNA
200T8REV

(SEQ ID NO: 25)

GGATCCACTAGTAACGKCCGCCAGTGTGCTGGAATTCKCCCTTAAG
CAGTGGTAACAACGCAGAGTACTTTTTTTTTTTTTTTTTTTTTTTT
TTTTAAAMAATTTTTTTWTWTTGAGARAASWCACAVACWCTGAGKG
GYTYYCCTTCWAAACAGCTCAMAAMCCAAAAAWSTGCYTCCCCATT
TKTTSWCATTGCYWYATCRAGGGTAGCRGGGGGTTKWTTTTTTGGC
TCCCCCCACCCCCATCTMAARAAGAGAAAAGGGGGYCCTKKTTTTW
YTTCMCAGAGTGTCKRGRRGGTTCCCCCYCCTSTCATTWTTACTRW
MAAAAATCGTTCTGWCACAGTGATTTSYCYTTCCCCCGCGACTCTG
CGTTTTYMCCACTKTTWAGGGGMRAWTTCTGAAGATWTCYATCACA
CTGGGGGCCGCTAGAGCATACATCTAGGGGGCCCAATNCNCNNNNN
NGNGAGTGGTWTCAAGKBKT
200T9REV

(SEQ ID NO: 26)

GATCCACTAKTAACGGCCGCCAGTGTGCTGGAATTYTCCCTTAAGC
AGTGGTAACAACGCAGAGTACGCGGG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

NAAANAANTTGGCTGGGCTGAAGG
200T10REV

(SEQ ID NO: 27)

CCCCDABNCGSCCGCCANTGKSCGGGNNTKMACBNNBSGGCGGGSS
KGCGGCGCNGAGKTCGGGGGGGGGGGTTGGGGGTGGTGGAAANAAA
TTTTTWTTATNNNNNNNTTNAWMTTTTTTTTTTNNNNTNTTNTTTT
TTTTTTTTNTTTTTNNKTNTTTTTTTNNTNNTNTTTTTTTTNNTNT
TNNTTGGNTNCNGGNNNTTTTNNNNTTNTTGTTGGGTGSGGCGCGG
SSSSSSCCCSSGCNAAATANNTTCGGGGTGGTGGGGGTGTTGGTNN
NNTKGGKTGTNNNNNTTTATTTGTTTTNNNTNNTTTKGTTGTGTGK
GKKGTGGTGGGGGGGGGGGGGSGGCGGGTGGG
200T11REV

(SEQ ID NO: 28)

TCCACTAGTAACGGCCGCCAGTGTGCTGGAATTCGCMNTTAAGCAG
TGGTAACAACGCAGAGTACTTTT

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

CCCGCGTANTTTGGGTTGGTAAAAATGTTTAAGGCGGGAATTTTNA
GATATAATAAAATGGGGGGCNTTTNNAATTAATTTTAGAGGGSCCA
TTACCCTATAATNANTCNTNTTATAATTWTATTNTNGGGAGTGRTS
TKTATAAAAATGTANNNTNGGNAAAAAATAGANGNTNAAAAAAATA
AATTNNGNKTTTGTCGNANNTNNNCNYTTTTNCNNANNTAGGNGKN
NTTNANGAAGNGGGNNGGNNNCCG
200T12REV

(SEQ ID NO: 29)

TCGGATCCACTAGTAACGGCCGCCAGTGTGCTGGAATTCGCCCTTA
AGCAGTGGTAACAACGCAGAGTACGCGGGTGCTGTCACTGGGAACC
TGGACGACTTCTCACTTGAAAACTTGCACCACCTCATGCCTTCCCT
TATCATAAGCCTGGGCAACCTCNTAATATTCTCCCCCTCACAATAG
AGACAAGGCAACCTGCAGTAAAAGTTTGAGCAAACTGGCCGGGCGC
GATGCCTCG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

NNNNNNNNANN
NNCTYTCCGTTGTWAACCACTGCTGTAAGGGGGCATATTTTCGTGS
NGNTTTTCCATCCCNNTKGGGNGGNCMGTTNGNNNAATTNNATTTG
TNGNAGGGGCCCCATTTTYCCCCCCTTTTNNNTGNANTCNNTNNTA
YGAYATTGTBASCGNGSCGCGGTGGTGKKRTTGWKGATNGGKTNNN
TTNNNCNTGNGGGANAAAACCCCCGTNGNGNGNNNWAAACCAAAAA
CANGTNNNTGGCCTTNGNNNNNNNNTCCNCNTTTTTNNCNN
200T13REV

(SEQ ID NO: 30)

GCGGRG [symbol] [symbol] [symbol] [symbol] [symbol]
AGCAGTGGTAACAACGCAGAGTACTTTTTTTTTTTTTTTTTTTTTT
TTTTCATTTGCTTAACTGGAAAGGCGTTTCCAATTGATTGAATAGC
AACGATAACTCCTCBTATAATGCCAGTAGCTATCTTTTTGTCTTGC
TCGTCTATTCGATCCCAGCCCGACAAAATGGTAATTATCAAAGTAA
GAACGATCGTGGTAATTGTTAACCSTGCATCCCAACTTCGATTGAT
TCGTCTGAGCCGAGTTATTTCTTGAGTTAATTCTTTAATTTCTTTC
TCTAAGAGTTTCCTTTGCTTCTCGATTATAGATAAAGATTGAGTTT
GCATTTTTTCTCTGATTTTTGAGCTGAGACAAGTTTTGAGTAAATG
CACCTTGCTGTGAGAAACAGTCAGTCTGATATCTACTTAAGCAGGT
AACGCTTTTTCATAAGCCTTAGCAAGTTTGATGTGGNATTCATTCT
GCCAATACAGCTCTCCGTTATAGCCAAAAGCGAACTTATCCCAATC
TTTAAGTTTGAGATGAACTAGCAAATTTCTGTTCACAATAAAGTTC
ATCATGTGTCTTGCTTGCCAATACTCTCCTGCAAAGTTTTCTCAAT
GAACTGCTGGATTGAATGCCACACCAGCAGCAGTNTAAKTGAATCC
CCATGACTTGNTTCGAGTCCAAAAGATGCACTTTTTATAGCCTGAA
ATTCATCAAAATKCCATTTGGMATCAAGTAAACGTTTTATCCCACT
CYTGCTGACCCCCCCAAAA
200T14REV

(SEQ ID NO: 31)

AGCTCGGATCCACTAGTAACGGCCGCCAGTGTGCTGGAATTCGCCM
TTAAGCAGTGGTAACAACGCAGAGTACTTTTTTTTTTTTTTTTTTT
TTTTTTTNTAAACVWAWAAATKTGTATWAAAAACGACAAARAAATT
TACATAGAAGATMCCATTAAAAACCAAAAACTAGATATTACAAAGC
TTCAATCCTTAAGTTAACTTATTTCTTACCGGTCTTCTTGGCGGAC
TTTTGTCCCWTATCTTGTTGTTCTTTCAACACCCCNHTAACGATCT
TTTKTTCTTCGAWAAAAAAAGCTCTGACAATTCTTYCCTTGACACA
GTTAGCACATCTAGAACCCCCGTAGCYCTTTGGACGGCCTTNGGGG
NCTTARAAACTTGAGCGNATTCCCTTGGTCTTAAAGTAGANAAACC
TTGTAAAGCATCACCACAGNCACCACACNTTGGTCTAAAANCGACC
TTCTTACGTTTTGGGMCAACTAATTTACCACMSKGGGGGTCTTTAA
CGCCCGTGNCCTTGNANANTGGGAAWGAATNNNGCCAAAAAAKNCN
CATTTGGACCGNATCACCGNCCTTT
200T15REV

(SEQ ID NO: 32)

GAGCTCGGATCCACTAGTAACGGCCGCCAGTGTGCTGGAATTCGCC
CTTAAGCAGTGGTAACAACGCAGAGTACGCGGGATACTTAAGTTCT
GTTAAAGTTAGCAACTACTTTCGTATTAAATCATCAAGAAAATCAC
ATAGGCCSTAAWWAT [symbol] [symbol] KCT

[symbol]

[symbol]

[symbol]

[symbol]

[symbol]

[symbol]

AGACCTAGAGACTGTCATAAGACTGAATTAGTCAGAAAGAGAAAAA
CAAATATCATTTGTTATATTATTAACACATAATGTGGAATCTAGAA
AAATGGTATAGATTATCTTTTTGCAACACAGNNGTTGAGACACCGA
TGTAGAGAACATCTGGATGCTGGKGGGGGAAGGAGAAAGGGGGTGA
TGAATTGGGAGATTGGGATTGATATNTNTNCCCTNCTNGCTGTAAG
TNGNTAACTAATGNGNACSTGCTGTNCNGCNCNNGGNNNTTTACTC
AGTGCTCTATGGTGACCTCNNNTGGNNNNGNNNTCBNNNCKGAGAG
GGGATATATGTAAACATATCGTTGNTTCCCTTTGGCTNTNGGGCCG
NNACTNNCCCNATTTTGTTNNGCCCCTTTTTTCCNNTNNVNGNTNN
TTTAAATGCGGGNNNNGAGGCG

An example of a sequence alignment of an Alu-like sequence containing fragment from a BSE cow (SEQ ID NOs:33 and 35) (see, sequence for clone 200T6REV) with a human sequence containing Alu repetitive sequences (SEQ ID NOs: 34 and 36) is provided below.

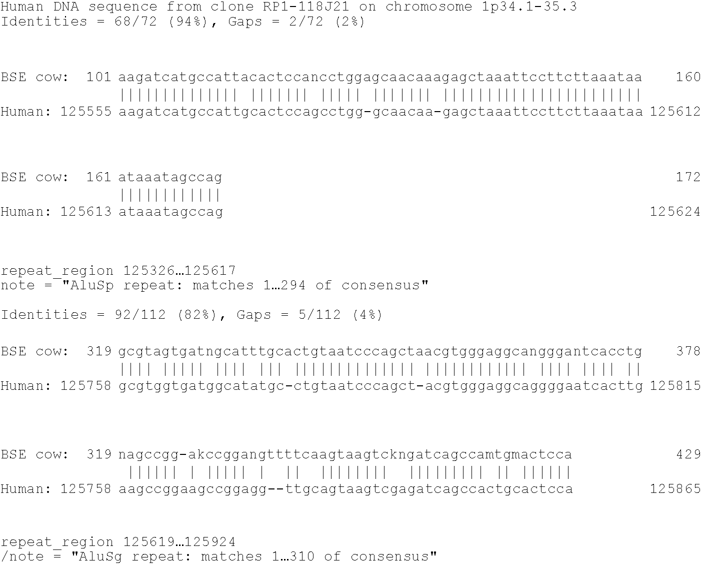

Examples 1 and 2 thus demonstrate the presence of repetitive sequences in amplicons generated from RNA in the serum of BSE cows.

The above examples are provided to illustrate the invention but not to limit its scope. Other variants of the invention will be readily apparent to one of ordinary skill in the art and are encompassed by the appended claims.

All publications, patents, and patent applications cited herein are hereby incorporated by reference for all purposes.

What is claimed is:

1. A method of detecting the presence of a target nucleic acid molecule in an acellular biological sample from an animal suspected of suffering from a disease, the method comprising:
providing the acellular biological sample;
contacting the sample with a polynucleotide probe that specifically hybridizes to a target nucleic acid molecule that comprises sequences from the animal's germline DNA, and
detecting a hybridization complex comprising the nucleic acid probe as indicative of the presence of the target nucleic acid molecule in the acellular biological sample.

2. The method of claim 1, wherein the target nucleic acid is an RNA molecule.

3. The method of claim 1, wherein the target nucleic acid comprises rearranged germline DNA.

4. The method of claim 3, wherein the rearranged germline DNA comprises Alu-like sequences.

5. The method of claim 3, wherein the rearranged germline DNA comprises SINES.

6. The method of claim 1, wherein the target nucleic acid molecule comprises chimeric sequences.

7. The method of claim 1, wherein the acellular biological sample is serum or plasma.

8. The method of claim 1, wherein the animal is suspected of having a chronic illness.

9. The method of claim 8, wherein the chronic illness a spongiform encephalopathy.

10. The method of claim 9, wherein the spongiform encephalopathy is bovine spongiform encephalomyelitis.

11. The method of claim 1, wherein the step of contacting includes a step of amplifying the target nucleic acid molecule.

12. The method of claim 11, wherein the step of amplification is carried out using a polymerase chain reaction (PCR).
